(19) United States
(12) Patent Application Publication
(10) Pub. No.: US 2026/0034154 A1
(43) Pub. Date: Feb. 5, 2026

(54) METHODS FOR TREATING NEWLY DIAGNOSED MYCOBACTERIUM AVIUM COMPLEX LUNG INFECTIONS

(71) Applicant: Insmed Incorporated, Bridgewater, NJ (US)

(72) Inventors: Kevin MANGE, Bridgewater, NJ (US); Dayton YUEN, Bridgewater, NJ (US); Zhanna JUMADILOVA, Bridgewater, NJ (US)

(73) Assignee: Insmed Incorporated, Bridgewater, NJ (US)

(21) Appl. No.: 19/355,944

(22) Filed: Oct. 10, 2025

Related U.S. Application Data
(63) Continuation of application No. 18/024,040.
(60) Provisional application No. 63/072,602, filed on Aug. 31, 2020.

(51) Int. Cl. A61K 31/7036; A61K 9/00; A61K 9/127; A61K 31/133; A61K 31/7048; A61K 31/7052; A61P 31/04

(52) U.S. Cl. CPC A61K 31/7036; A61K 9/0078; A61K 9/127; A61K 31/133; A61K 31/7048; A61K 31/7052; A61P 31/04

(57) ABSTRACT

Provided is a method for treating a newly diagnosed and untreated MAC lung infection comprising administering to the lungs of a patient for an administration period of at least about 6 months, a pharmaceutical composition comprising amikacin, or a pharmaceutically acceptable salt thereof, The pharmaceutical composition is aerosolized via a nebulizer to provide an aerosolized pharmaceutical composition comprising a mixture of free amikacin, or a pharmaceutically acceptable salt thereof, and liposomal complexed amikacin, or a pharmaceutically acceptable salt thereof, and the aerosolized pharmaceutical composition is administered via the nebulizer to the lungs of the patient once daily in a single dosing session. The treating comprises improving a respiratory symptom score as defined by one or more patient reported outcomes, during or subsequent to the administration period.

### CROSS-REFERENCE TO RELATED APPLICATION

This application is a continuation of U.S. patent application Ser. No. 18/024,040, filed Feb. 28, 2023, which is a national phase entry of International Application No. PCT/US2021/048199, filed Aug. 30, 2021, which claims priority from U.S. Provisional Patent Application No. 63/072,602, filed Aug. 31, 2020, the disclosure of each of which is incorporated by reference in its entirety.

### BACKGROUND OF THE INVENTION

Nontuberculous mycobacterial (NTM) lung disease caused by Mycobacterium avium complex (MAC) is a potentially life-threatening and progressively destructive disease that is associated with symptoms of productive cough, fatigue, shortness of breath, fever, weight loss, lung function decline, and hemoptysis. It often complicates other chronic debilitating underlying lung diseases such as bronchicctasis or COPD. When NTM lung disease occurs in patients without underlying lung comorbidities, it has been implicated in progressive lung disease.

The current treatment of MAC lung disease is primarily with a multidrug regimen based on the treatment of tuberculosis. The recommendation for MAC lung disease is a 3-drug antibiotic regimen that includes a macrolide, ethambutol, and a rifamycin. The regimen is administered for 12 months beyond culture conversion to negative, although the duration of antimicrobial therapy often exceeds 18 months. See Johnson and Odell. Nontuberculous mycobacterial pulmonary infections. J Thoracic Dis. 2014; 6(3): 210-220, incorporated herein by reference in its entirety. Intravenous amikacin or intramuscular streptomycin are recommended for patients with fibrocavitary disease or severe nodular/bronchiectatic disease; however, the optimal treatment has yet to be established. Intravenous aminoglycoside use is limited by poor penetration into lung tissue, poor uptake by alveolar macrophages (see Zhang et al. Afatinib decreases p-glycoprotein expression to promote adriamycin toxicity of A549T Cells. J Cell Biochem. 2018; 119, 414-423, incorporated herein by reference in its entirety), and the potential for ototoxicity, loss of balance, and impaired kidney function with high or prolonged systemic exposure (see Kovacevic et al. Therapeutic monitoring of amikacin and gentamicin in critically and noncritically ill patients. J Basic Clinical Pharm. 2016; 7:65-69; Rybak et al. Prospective evaluation of the effect of an aminoglycoside dosing regimen on rates of observed nephrotoxicity and ototoxicity. Antimicrobial Agents and Chemotherapy. 1999; 43(7): 1549-1555; each of which is incorporated herein by reference in its entirety).

There remains an unmet medical need for patients with newly diagnosed MAC lung infections who have not started treatment, specifically for a treatment method that includes a patient reported outcome as a measure of efficacy. The present invention addresses this and other needs.

### SUMMARY OF THE INVENTION

The present application provides a method for treating a newly diagnosed and untreated Mycobacterium avium complex (MAC) lung infection in a patient in need of treatment.

In one embodiment, the method comprises:
(i) administering to the lungs of the patient for an administration period of at least about 6 months, a pharmaceutical composition comprising amikacin, or a pharmaceutically acceptable salt thereof, encapsulated in a plurality of liposomes, wherein the lipid component of the plurality of liposomes consists of dipalmitoylphosphatidylcholine (DPPC) and cholesterol, wherein administering to the lungs of the patient comprises aerosolizing the pharmaceutical composition via a nebulizer to provide an aerosolized pharmaceutical composition comprising a mixture of free amikacin, or a pharmaceutically acceptable salt thereof, and liposomal complexed amikacin, or a pharmaceutically acceptable salt thereof, and administering the aerosolized pharmaceutical composition via the nebulizer to the lungs of the patient once daily in a single dosing session during the administration period, and
(ii) administering to the patient during the administration period a macrolide antibiotic and ethambutol,
wherein the treating comprises improving a respiratory symptom score as measured by a patient reported outcome (PRO) during the administration period or subsequent to the administration period, compared to the respiratory symptom score of the patient measured at a baseline, i.e., prior to the administration period. In a further embodiment, the respiratory symptom score is the respiratory domain score of the Quality of Life Questionnaire-Bronchiectasis (QOL-B) or a modified version thereof. In one embodiment, the respiratory symptom score is the respiratory domain score of a QOL-B that has been modified by replacing the term “bronchiectasis” with “lung condition”.

In a further embodiment, the respiratory symptom score is improved about one month after the administration period. In a further embodiment, the administration period is from about 12 to about 24 months. In another embodiment, the respiratory symptom score is improved at least about one month after the administration period. In still a further embodiment, the respiratory symptom score is improved at about one to about three months after the administration period.

The administration period, in one embodiment, is from about 12 months to about 36 months.

In one embodiment, the treating comprises achieving a MAC sputum culture conversion in the patient during or subsequent to the administration period. The MAC sputum culture conversion, in one embodiment, is defined as two consecutive negative MAC sputum cultures. In one embodiment, the patient achieves the MAC sputum culture conversion during the administration period. In one embodiment, the patient achieves the MAC sputum culture conversion about three months subsequent to the administration period. In still a further embodiment, the patient achieves the MAC sputum culture conversion at least about three months after the administration period. In still a further embodiment, the patient achieves the MAC sputum culture conversion from about three to about six months after the administration period. In still a further embodiment, the patient achieves the MAC sputum culture conversion from about three to about twelve months after the administration period.

In one embodiment, the administration period is from about six months to about twelve months. In another embodiment, the administration period is about 12 months. In another embodiment, the administration period is from about 12 to about 24 months. In another embodiment, the administration period is from about 12 to about 36 months. In one embodiment, the administration period is 12 months and the respiratory symptom score is assessed about one month subsequent to the administration period, i.e., at about 13 months from initiating treatment.

In one embodiment of the method disclosed herein, the pharmaceutical composition comprises from about 500 mg to about 650 mg amikacin, or pharmaceutically acceptable salt thereof. In a further embodiment, the amikacin or pharmaceutically acceptable salt thereof is amikacin sulfate.

In one embodiment of the method disclosed herein, the pharmaceutical composition comprises about 590 mg amikacin, or pharmaceutically acceptable salt thereof. In a further embodiment, the amikacin or pharmaceutically acceptable salt thereof is amikacin sulfate.

In one embodiment of the method disclosed herein, the pharmaceutical composition comprises about 70 mg/mL amikacin sulfate; about 30 to about 35 mg/mL DPPC; and about 15 to about 17 mg/mL cholesterol. In a further embodiment, the pharmaceutical composition comprises about 70 mg/mL amikacin sulfate; about 32 mg/mL DPPC; and about 16 mg/mL cholesterol.

In one embodiment of the method disclosed herein, the pharmaceutical composition is an aqueous dispersion, with a volume of from about 8 mL to about 10 mL. In a further embodiment, the volume of the pharmaceutical composition is about 8.4 mL.

In one embodiment of the method disclosed herein, the macrolide antibiotic is azithromycin.

In one embodiment of the method disclosed herein, the patient has a non-cavitary MAC lung disease. In another embodiment, the patient has a non-cystic fibrosis lung disease.

### BRIEF DESCRIPTION OF THE FIGURES

FIG. 1 is a subject eligibility flowchart for the studies set forth in Examples 1 and 2. Refractory=MAC treatment≥6 months and culture remains positive; Relapse=successful treatment, defined as cessation of treatment and documented negative culture, with a return to positive culture less than 6 months after cessation of treatment.

FIG. 2 is a schematic diagram of the study design according to Example 1. ALIS: amikacin liposome inhalation suspension; AZI: azithromycin 250 mg; ELC: empty liposome control (matching placebo for ALIS); EOS: end of study; EOT: end of treatment; ETH: ethambutol 15 mg/kg; R: randomization.

FIG. 3 is a schematic diagram of the study design according to Example 2. ALIS: amikacin liposome inhalation suspension; AZI: azithromycin 250 mg; ELC: empty liposome control (matching placebo for ALIS); EOS: end of study; EOT: end of treatment; ETH: ethambutol 15 mg/kg; R: randomization.

### DETAILED DESCRIPTION OF THE INVENTION

The term “about,” as used herein, refers to plus or minus ten percent of the object that “about” modifies.

The term “treating” in one embodiment, includes: (1) preventing or delaying the appearance of clinical symptoms of the state, disorder or condition developing in the subject that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (e.g., causing regression of the state, disorder or condition or at least one of its clinical or subclinical symptoms). In one embodiment, “treating” refers to inhibiting the state, disorder or condition (e.g., arresting, reducing or delaying the development of the disease, or a relapse thereof in case of maintenance treatment, of at least one clinical or subclinical symptom thereof). In another embodiment, “treating” refers to relieving the condition (for example, by causing regression of the state, disorder or condition or at least one of its clinical or subclinical symptoms). The benefit to a subject to be treated is either statistically significant as compared to the state or condition of the same subject before the treatment, or as compared to the state or condition of an untreated control subject, or the benefit is at least perceptible to the subject or to the physician. Treating, in one aspect described herein, refers to the improvement of a respiratory symptom score as measured by a patient reported outcome (PRO) during the administration period or subsequent to the administration period (e.g., one month after end of the administration period). For example, the respiratory symptom score in one embodiment, comprises the respiratory domain score of the Quality of Life Questionnaire-Bronchiectasis (QOL-B) or a modified version thereof. In one embodiment, the respiratory symptom score comprises the respiratory domain score of the QOL-B that has been modified to replace the term “bronchiectasis” with “lung condition”. In another embodiment, the respiratory symptom score comprises a subset of the respiratory domain score of the QOL-B or a modified version thereof, i.e., a subset of the nine symptom queries in the QOL-B. In a further embodiment, the respiratory the respiratory domain score of the QOL-B is modified to replace the term “bronchiectasis” with “lung condition”.

“A newly diagnosed and untreated Mycobacterium avium complex (MAC) lung infection”, as used herein, refers to either:
(i) an untreated, initially diagnosed MAC lung infection based on a positive MAC sputum culture where the patient has not been previously treated for the MAC lung infection, or
(ii) an untreated, subsequent MAC lung infection, characterized as follows: a subject initially ceasing MAC lung infection treatment based on a documented negative MAC sputum culture, with a return to a positive MAC sputum culture more than 6 months after cessation of the prior MAC lung infection treatment.

“Culture conversion”, or “MAC culture conversion”, as used herein, refers to at least two consecutive negative MAC sputum cultures, spaced about one month apart, or about 30 days apart. In one embodiment, culture conversion refers to two consecutive negative MAC sputum cultures, spaced about one month, or about 30 days apart. In one embodiment, culture conversion refers to three consecutive negative MAC sputum cultures, spaced about one month, or about 30 days apart.

“Effective amount” means an amount of amikacin, or a pharmaceutically acceptable salt thereof, used in the present invention sufficient to result in the desired therapeutic response. The effective amount of the pharmaceutical composition provided herein comprises both free and liposomal complexed amikacin, or a pharmaceutically acceptable salt thereof. For example, the liposomal complexed amikacin, or a pharmaceutically acceptable salt thereof, in one embodiment, comprises amikacin, or a pharmaceutically acceptable salt thereof, complexed with the liposome bilayer, encapsulated within the liposome bilayer, or a combination thereof.

“Liposomal dispersion” refers to a solution or suspension comprising a plurality of liposomes.

An “aerosol,” as used herein, is a gaseous suspension of liquid particles. The aerosol provided herein comprises particles of the liposomal dispersion.

A “nebulizer” or an “aerosol generator” is a device that converts a liquid into an aerosol of a size that can be inhaled into the respiratory tract. Pneumonic, ultrasonic, electronic nebulizers, e.g., passive electronic mesh nebulizers, active electronic mesh nebulizers and vibrating mesh nebulizers are amenable for use with the invention if the particular nebulizer emits an aerosol with the required properties, and at the required output rate.

The process of pneumatically converting a bulk liquid into small droplets is called atomization. The operation of a pneumatic nebulizer requires a pressurized gas supply as the driving force for liquid atomization. Ultrasonic nebulizers use electricity introduced by a piezoelectric element in the liquid reservoir to convert a liquid into respirable droplets. Various types of nebulizers are described in Respiratory Care, Vol. 45, No. 6, pp. 609-622 (2000), the disclosure of which is incorporated herein by reference in its entirety. The terms “nebulizer” and “aerosol generator” are used interchangeably throughout the specification. “Inhalation device,” “inhalation system” and “atomizer” are also used in the literature interchangeably with the terms “nebulizer” and “aerosol generator.”

“Mass median diameter” or “MMD” is determined by laser diffraction or impactor measurements, and is the average particle diameter by mass.

“Mass median aerodynamic diameter” or “MMAD” is normalized regarding the aerodynamic separation of aqua aerosol droplets and is determined impactor measurements, e.g., the Anderson Cascade Impactor (ACI) or the Next Generation Impactor (NGI). The gas flow rate, in one embodiment, is 28 Liter per minute by the Anderson Cascade Impactor (ACI) and 15 Liter per minute by the Next Generation Impactor (NGI). “Geometric standard deviation” or “GSD” is a measure of the spread of an aerodynamic particle size distribution.

A “pharmaceutically acceptable salt” includes both acid and base addition salts. In one embodiment, a pharmaceutically acceptable salt is a pharmaceutically acceptable acid addition salt which retains the biological effectiveness and properties of the free base, and which is not biologically or otherwise undesirable. A pharmaceutically acceptable acid addition salt may be formed with an inorganic acid, such as, but not limited to, hydrochloric acid (HCl), hydrobromic acid, sulfuric acid, nitric acid, phosphoric acid, or may be formed with an organic acid, such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecyl sulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid (e.g., as lactate), lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, acetic acid (e.g., as acetate), tartaric acid, thiocyanic acid, p-toluenesulfonic acid, trifluoroacetic acid (TFA), and undecylenic acid. In one embodiment, the pharmaceutically acceptable salt is a HCl, TFA, lactate, or acetate salt. In another embodiment, the pharmaceutically acceptable salt is a sulfate salt.

Throughout the present specification, numerical ranges are provided for certain quantities. It is to be understood that these ranges comprise all subranges therein. Thus, the range “from 50 to 80” includes all possible ranges therein (e.g., from 51 to 79, from 52 to 78, from 53 to 77, from 54 to 76, from 55 to 75, from 60 to 70, etc.). Furthermore, all values within a given range may be an endpoint for the range encompassed thereby (e.g., the range of from 50 to 80 includes the ranges with endpoints such as from 55 to 80, from 50 to 75, etc.).

The present application provides a method for treating a newly diagnosed and untreated Mycobacterium avium complex (MAC) lung infection in a patient in need of treatment. In one embodiment, the method comprises:
(i) administering to the lungs of the patient for an administration period of at least about 6 months, a pharmaceutical composition comprising amikacin, or a pharmaceutically acceptable salt thereof, encapsulated in a plurality of liposomes, wherein the lipid component of the plurality of liposomes consists of dipalmitoylphosphatidylcholine (DPPC) and cholesterol,
wherein administering to the lungs of the patient comprises aerosolizing the pharmaceutical composition via a nebulizer to provide an aerosolized pharmaceutical composition comprising a mixture of free amikacin, or a pharmaceutically acceptable salt thereof, and liposomal complexed amikacin, or a pharmaceutically acceptable salt thereof, and administering the aerosolized pharmaceutical composition via the nebulizer to the lungs of the patient once daily in a single dosing session during the administration period, and
(ii) administering to the patient during the administration period a macrolide antibiotic and ethambutol,
wherein the treating comprises improving a respiratory symptom score as measured by a patient reported outcome (PRO) during the administration period or subsequent to the administration period, compared to the respiratory symptom score of the patient measured at a baseline, i.e., prior to the administration period. For example, the respiratory symptom score in one embodiment, comprises the respiratory domain score of the Quality of Life Questionnaire-Bronchiectasis (QOL-B) or a modified version thereof. In one embodiment, the respiratory symptom score comprises the respiratory domain score of the QOL-B that has been modified to replace the term “bronchiectasis” with “lung condition”. In another embodiment, the respiratory symptom score comprises a subset of the respiratory domain score of the QOL-B or a modified version thereof, i.e., a subset of the nine symptom queries in the QOL-B. In a further embodiment, the respiratory the respiratory domain score of the QOL-B is modified to replace the term “bronchiectasis” with “lung condition”.

In a further embodiment, the treating comprises improving the QOL-B respiratory domain score, for the patient at twelve months or thirteen months after the treating as commenced, as compared to the QOL-B respiratory domain score of the patient prior to the administration period.

In one embodiment, the newly diagnosed and untreated MAC lung infection is defined as an untreated, initially diagnosed MAC lung infection based on a positive MAC sputum culture. Namely, the patient in need of treatment has an initial (first) and current diagnosis of a MAC lung infection based on a positive sputum culture for MAC, and the patient has not started treatment for the current MAC lung infection.

In another embodiment, the newly diagnosed and untreated MAC lung infection is an untreated, subsequent MAC lung infection, characterized as follows: a subject initially ceasing MAC lung infection treatment based on a documented negative MAC sputum culture, with a return to a positive MAC sputum culture greater than or equal to 6 months after cessation of the prior MAC lung infection treatment (e.g., about 6 months, about 6 months to about 12 months, or about 6 months to about 9 months) after the cessation of the treatment of the previously diagnosed MAC lung infection.

In a further embodiment, prior to the newly positive sputum culture for MAC, the patient presents at least one negative sputum culture for MAC at least 6 months after the cessation of the treatment of the previously diagnosed MAC lung infection.

In one embodiment, the previously diagnosed MAC lung infection is treated, and the treatment of the previously diagnosed MAC lung infection is ceased when a MAC sputum culture conversion in the patient is achieved, wherein the MAC sputum culture conversion is defined as at least two consecutive negative MAC sputum cultures, e.g., the treatment of the previously diagnosed MAC lung infection is ceased when the second of the two consecutive negative MAC sputum cultures is achieved; and the second of the two consecutive negative MAC sputum cultures returns to the newly positive sputum culture for MAC at least 6 months (e.g., about 6 months, about 6 months to about 12 months, or about 6 months to about 9 months) after the cessation of the treatment of the previously diagnosed MAC lung infection. The MAC sputum cultures, in one embodiment, are obtained from the patient about 30 days apart.

In one embodiment, the patient in need of treatment was previously diagnosed with a first MAC lung infection and the patient has a second and current diagnosis of a MAC lung infection based on a newly positive sputum culture for MAC, and the patient has not started treatment for the second and current MAC lung infection.

In another embodiment, the previously diagnosed MAC lung infection is a second diagnosed MAC lung infection, and the patient in need of treatment has a third and current diagnosis of a MAC lung infection based on a newly positive sputum culture for MAC, and the patient has not started treatment for the third and current MAC lung infection.

In still another embodiment, the previously diagnosed MAC lung infection is a third diagnosed MAC lung infection, and the patient in need of treatment has a fourth and current diagnosis of a MAC lung infection based on a newly positive sputum culture for MAC, and the patient has not started treatment for the fourth and current MAC lung infection.

In one embodiment, the patient has a non-cavitary MAC lung disease.

In another embodiment, the patient has a non-cystic fibrosis lung disease.

In another embodiment, the patient has a non-cavitary, non-cystic fibrosis lung disease.

In one embodiment, the pharmaceutical composition, as provided herein, is a liposomal dispersion comprising liposomal complexed amikacin, or a pharmaceutically acceptable salt thereof, wherein amikacin, or a pharmaceutically acceptable salt thereof, is (i) complexed with the liposome bilayer, (ii) encapsulated within the liposome bilayer, or (iii) a combination thereof. As provided above, embodiments of liposomal complexed amikacin, or a pharmaceutically acceptable salt thereof include embodiments where the amikicin or pharmaceutically acceptable salt thereof is encapsulated in a plurality of liposomes. Liposomes are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer), or a combination thereof. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer, while the hydrophilic “heads” orient towards the aqueous phase. Amikacin, or a pharmaceutically acceptable salt thereof, may be in the aqueous phase, or in the hydrophobic bilayer phase, or at the interfacial headgroup region of the liposomal bilayer. In one embodiment, the pharmaceutical composition comprises amikacin, or a pharmaceutically acceptable salt thereof encapsulated within the bilayers of a plurality of liposomes.

In one embodiment, the amikacin or pharmaceutically acceptable salt thereof is amikacin sulfate. In another embodiment, the amikacin or pharmaceutically acceptable salt thereof is amikacin disulfate.

In one embodiment, prior to nebulization of the pharmaceutical composition, at least about 5%, at least about 10%, at least about 20%, at least about 25%, at least about 50%, at least about 70%, at least about 80%, at least about 85%, at least about 90%, at least about 95%, at least about 97% by weight of amikacin, or a pharmaceutically acceptable salt thereof, present in the composition is liposomal complexed. In a further embodiment, the liposomal complexed amikacin, or a pharmaceutically acceptable salt thereof comprises the amikacin, or pharmaceutically acceptable salt thereof encapsulated in a plurality of liposomes. In still a further embodiment, the amikacin or pharmaceutically acceptable salt thereof is amikacin sulfate.

In another embodiment, at least about 97% by weight of amikacin, or a pharmaceutically acceptable salt thereof, present in the composition is liposomal complexed prior to nebulization. In a further embodiment, the liposomal complexed amikacin, or a pharmaceutically acceptable salt thereof comprises the amikacin, or pharmaceutically acceptable salt thereof encapsulated in a plurality of liposomes. In still a further embodiment, the amikacin or pharmaceutically acceptable salt thereof is amikacin sulfate.

In another embodiment, prior to nebulization of the pharmaceutical composition, about 70% to about 100%, about 80% to about 99%, about 85% to about 99%, about 90% to about 99%, about 95% to about 99%, or about 96% to about 99% by weight of amikacin, or a pharmaceutically acceptable salt thereof, present in the composition is liposomal complexed. In another embodiment, prior to nebulization of the pharmaceutical composition, about 95% to about 99% by weight of amikacin, or a pharmaceutically acceptable salt thereof, present in the composition, is liposomal complexed. In a further embodiment, the liposomal complexed amikacin, or a pharmaceutically acceptable salt thereof, comprises the amikacin, or pharmaceutically acceptable salt thereof, encapsulated in a plurality of liposomes. In still a further embodiment, the amikacin or pharmaceutically acceptable salt thereof is amikacin sulfate.

The percentage of liposomal complexed amikacin, or a pharmaceutically acceptable salt thereof may be measured, in one embodiment, by a method described in U.S. Pat. No. 4,752,425, the disclosure of which is incorporated herein by reference in its entirety. The percentage of liposomal complexed amikacin, or a pharmaceutically acceptable salt thereof is measured, in another embodiment, by a method described in U.S. Pat. No. 9,566,234, the disclosure of which is incorporated herein by reference in its entirety.

In some embodiments, the lipid component of the plurality of liposomes consists of DPPC and cholesterol, with a DPPC/cholesterol molar ratio in the range of from about 19:1 (DPPC:cholesterol) to about 1:1 (DPPC:cholesterol), or from about 9:1 (DPPC:cholesterol) to about 1:1 (DPPC:cholesterol), or from about 4:1 (DPPC:cholesterol) to about 1:1 (DPPC:cholesterol), or from about 2:1 (DPPC:cholesterol) to about 1:1 (DPPC:cholesterol). In a further embodiment, the DPPC and cholesterol have a molar ratio of about 2:1 (DPPC:cholesterol).

In one embodiment, the weight ratio of the lipid (i.e., lipid component) to amikacin or a pharmaceutically acceptable salt thereof in the pharmaceutical composition provided herein is from about 0.1:1 (lipid:amikacin or a pharmaceutically acceptable salt thereof) to about 1:1 (lipid:amikacin or a pharmaceutically acceptable salt thereof), e.g., about 0.1:1 (lipid:amikacin or a pharmaceutically acceptable salt thereof), about 0.2:1 (lipid:amikacin or a pharmaceutically acceptable salt thereof), about 0.3:1 (lipid:amikacin or a pharmaceutically acceptable salt thereof), about 0.4:1 (lipid:amikacin or a pharmaceutically acceptable salt thereof), about 0.5:1 (lipid:amikacin or a pharmaceutically acceptable salt thereof), about 0.6:1 (lipid:amikacin or a pharmaceutically acceptable salt thereof), about 0.7:1 (lipid:amikacin or a pharmaceutically acceptable salt thereof), about 0.8:1 (lipid:amikacin or a pharmaceutically acceptable salt thereof), about 0.9:1 (lipid:amikacin or a pharmaceutically acceptable salt thereof), or about 1:1 (lipid:amikacin or a pharmaceutically acceptable salt thereof). In another embodiment, the lipid-to-amikacin or a pharmaceutically acceptable salt thereof weight ratio is from about 0.3:1 (lipid:amikacin or a pharmaceutically acceptable salt thereof) to about 1:1 (lipid:amikacin or a pharmaceutically acceptable salt thereof). In another embodiment, the lipid-to-amikacin or a pharmaceutically acceptable salt thereof weight ratio is from about 0.5:1 (lipid:amikacin or a pharmaceutically acceptable salt thereof) to about 0.9:1 (lipid:amikacin or a pharmaceutically acceptable salt thereof). In another embodiment, the lipid-to-amikacin or a pharmaceutically acceptable salt thereof weight ratio is from about 0.6:1 (lipid:amikacin or a pharmaceutically acceptable salt thereof) to about 0.8:1 (lipid:amikacin or a pharmaceutically acceptable salt thereof). In another embodiment, the lipid-to-amikacin or a pharmaceutically acceptable salt thereof weight ratio is from about 0.65:1 (lipid:amikacin or a pharmaceutically acceptable salt thereof) to about 0.75:1 (lipid:amikacin or a pharmaceutically acceptable salt thereof). In another embodiment, the lipid-to-amikacin or a pharmaceutically acceptable salt thereof weight ratio is about 0.7:1 (lipid:amikacin or a pharmaceutically acceptable salt thereof). In a further embodiment, the amikacin or pharmaceutically acceptable salt thereof is amikacin sulfate.

In one embodiment, the liposomes provided herein are small enough to effectively penetrate patient mucus and biofilms. In some embodiments, the mean diameter of the plurality of liposomes, as measured by light scattering is from about 100 nm to about 400 nm, or from about 150 nm to about 350 nm, or from about 200 nm to about 300 nm, or from about 250 nm to about 300 nm. In a further embodiment, the mean diameter of the plurality of liposomes, as measured by light scattering is from about 150 nm to about 350 nm. In still a further embodiment, the mean diameter of the plurality of liposomes, as measured by light scattering is from about 260 nm to about 280 nm.

In one embodiment, the liposomal compositions described herein are manufactured by one of the methods set forth in U.S. Patent Application Publication No. 2013/0330400, U.S. Pat. No. 7,718,189, PCT Application Publication WO 2019/191627, and PCT Application Publication WO 2019/213398, each of which is incorporated by reference in its entirety for all purposes.

In one embodiment, the method described herein comprises administering to the lungs of the patient a pharmaceutical composition comprising amikacin, or a pharmaceutically acceptable salt thereof (e.g., amikacin sulfate), encapsulated in a plurality of liposomes by inhalation via a nebulizer. In one embodiment, the amount of amikacin, or a pharmaceutically acceptable salt thereof provided in the composition is about 450 mg, about 500 mg, about 550 mg, about 560 mg, about 570 mg, about 580 mg, about 590 mg, about 600 mg or about 610 mg. In another embodiment, the amount of amikacin, or a pharmaceutically acceptable salt thereof provided in the composition is from about 500 mg to about 600 mg, or from about 500 mg to about 650 mg, or from about 525 mg to about 625 mg, or from about 550 mg to about 600 mg. In another embodiment, the amount of amikacin, or a pharmaceutically acceptable salt thereof provided in the composition is from about 500 mg to about 650 mg. In another embodiment, the amount of amikacin, or a pharmaceutically acceptable salt thereof provided in the composition is about 590 mg. In a further embodiment, the amikacin or pharmaceutically acceptable salt thereof is amikacin sulfate.

In one embodiment, the pharmaceutical composition is an aqueous liposomal dispersion. In a further embodiment, the volume of the pharmaceutical composition is from about 8 mL to about 10 mL. In still a further embodiment, the volume of the pharmaceutical composition is about 8.4 mL.

In one embodiment, the amount of amikacin, or a pharmaceutically acceptable salt thereof administered to the patient is about 560 mg and is provided in an 8.4 mL composition. In a further embodiment, the amikacin or pharmaceutically acceptable salt thereof is amikacin sulfate. In another embodiment, the amount of amikacin, or a pharmaceutically acceptable salt thereof administered to the patient is about 590 mg and is provided in an 8.4 mL composition. In a further embodiment, the amikacin or pharmaceutically acceptable salt thereof is amikacin sulfate. In another embodiment, the amount of amikacin, or a pharmaceutically acceptable salt thereof administered to the patient is about 600 mg and is provided in an 8.4 mL composition. In a further embodiment, the amikacin or pharmaceutically acceptable salt thereof is amikacin sulfate.

In one embodiment, the pharmaceutical composition provided herein comprises about 60 to about 90 mg/mL, about 60 to about 80 mg/mL, about 65 to about 85 mg/mL, about 70 to about 80 mg/mL, or about 70 to about 75 mg/mL amikacin, or a pharmaceutically acceptable salt thereof. In a further embodiment, about 96% to about 99% by weight of amikacin, or a pharmaceutically acceptable salt thereof, present in the composition is liposomal complexed prior to nebulization. In a further embodiment, the liposomal complexed amikacin, or a pharmaceutically acceptable salt thereof comprises the amikacin, or pharmaceutically acceptable salt thereof encapsulated in a plurality of liposomes. In still a further embodiment, the amikacin, or pharmaceutically acceptable salt thereof is amikacin sulfate. In another embodiment, the pharmaceutical composition comprises about 60 to about 80 mg/ml amikacin, or a pharmaceutically acceptable salt thereof. In a further embodiment, about 96% to about 99% by weight of amikacin, or a pharmaceutically acceptable salt thereof, present in the composition is liposomal complexed prior to nebulization. In a further embodiment, the liposomal complexed amikacin, or a pharmaceutically acceptable salt thereof comprises the amikacin, or pharmaceutically acceptable salt thereof encapsulated in a plurality of liposomes. In still a further embodiment, the amikacin, or pharmaceutically acceptable salt thereof is amikacin sulfate. In another embodiment, the pharmaceutical composition comprises about 70 to about 75 mg/mL amikacin, or a pharmaceutically acceptable salt thereof. In a further embodiment, about 96% to about 99% by weight of amikacin, or a pharmaceutically acceptable salt thereof, present in the composition is liposomal complexed prior to nebulization. In a further embodiment, the liposomal complexed amikacin, or a pharmaceutically acceptable salt thereof comprises the amikacin, or pharmaceutically acceptable salt thereof encapsulated in a plurality of liposomes. In still a further embodiment, the amikacin, or pharmaceutically acceptable salt thereof is amikacin sulfate. In another embodiment, the pharmaceutical composition comprises about 70 mg/mL amikacin, or a pharmaceutically acceptable salt thereof. In a further embodiment, about 96% to about 99% by weight of amikacin, or a pharmaceutically acceptable salt thereof, present in the composition is liposomal complexed prior to nebulization. In a further embodiment, the liposomal complexed amikacin, or a pharmaceutically acceptable salt thereof comprises the amikacin, or pharmaceutically acceptable salt thereof encapsulated in a plurality of liposomes. In still a further embodiment, the amikacin, or pharmaceutically acceptable salt thereof is amikacin sulfate.

In one embodiment, the pharmaceutical composition provided herein comprises about 25 to about 40 mg/mL DPPC. In another embodiment, the pharmaceutical composition provided herein comprises about 30 to about 40 mg/mL DPPC. In another embodiment, the pharmaceutical composition provided herein comprises about 35 to about 40 mg/mL DPPC. In another embodiment, the pharmaceutical composition provided herein comprises about 30 to about 35 mg/mL DPPC. In another embodiment, the pharmaceutical composition provided herein comprises about 32 to about 35 mg/mL DPPC.

In one embodiment, the pharmaceutical composition provided herein comprises about 10 to about 20 mg/mL cholesterol. In another embodiment, the pharmaceutical composition provided herein comprises from about 15 to about 20 mg/mL cholesterol. In another embodiment, the pharmaceutical composition provided herein comprises from about 15 to about 17 mg/mL cholesterol. In another embodiment, the pharmaceutical composition provided herein comprises from about 16 to about 17 mg/mL cholesterol.

In even another embodiment, the pharmaceutical composition provided herein comprises about 25 to about 40 mg/mL DPPC and from about 10 to about 20 mg/ml cholesterol. In a further embodiment, the pharmaceutical composition provided herein comprises about 26 to about 39 mg/mL DPPC and from about 11 to about 19 mg/mL cholesterol. In even a further embodiment, the pharmaceutical composition comprises from about 60 to about 80 mg/mL amikacin or amikacin sulfate. In yet even a further embodiment, the pharmaceutical composition comprises from about 65 to about 75 mg/mL amikacin or amikacin sulfate.

In one embodiment, the pharmaceutical composition provided herein comprises about 0.5% (w/w) to about 5% (w/w), about 0.5% (w/w) to about 3% (w/w), about 0.5% (w/w) to about 2% (w/w), about 0.75% (w/w), about 1.0% (w/w) about 1.25% (w/w), about 1.5% (w/w), about 1.75% (w/w), about 2.0% (w/w), or about 2.5% (w/w) NaCl. In another embodiment, the pharmaceutical composition provided herein comprises about 0.5% (w/w) to about 5% (w/w) NaCl. In another embodiment, the pharmaceutical composition provided herein comprises about 0.5% (w/w) to about 2% (w/w) NaCl. In another embodiment, the pharmaceutical composition provided herein comprises about 1.5% (w/w) NaCl.

In one embodiment, the pharmaceutical composition provided herein has a pH of about 6 to about 7. In another embodiment, the pharmaceutical composition provided herein has a pH of about 6.5.

In one embodiment, the pharmaceutical composition comprises amikacin sulfate. The density of the liposomal amikacin sulfate composition is about 1.05 gram/mL; and in one embodiment, approximately 8.4 grams of the liposomal amikacin sulfate composition per dose is present in the composition. In a further embodiment, the entire volume of the composition is administered to the patient in need thereof.

In one embodiment, the pharmaceutical composition is one of the compositions provided in Table 1, below.

TABLE 1
Exemplary Pharmaceutical Compositions

| Composition A (pH 6.0-7.0) | | Composition D (pH ~6.5) | | |
|---|---|---|---|---|
| Amikacin | 60-80 mg/mL | Amikacin | ~70 | mg/mL |
| Sulfate | | Sulfate | | |
| DPPC | 30-40 mg/mL | DPPC | ~32-35 | mg/mL |
| Cholesterol | 10-20 mg/mL | Cholesterol | ~16-17 | mg/mL |
| NaCl | 0.5%-5.0% | NaCl | ~1.5% | |
| Composition B (pH 6.0-7.0) | | Composition E (pH ~6.5) | | |
| Amikacin | 70-80 mg/mL | Amikacin | ~70 | mg/mL |
| Sulfate | | Sulfate | | |
| DPPC | 35-40 mg/mL | DPPC | ~30-35 | mg/mL |
| Cholesterol | 15-20 mg/mL | Cholesterol | ~15-17 | mg/mL |

| | | | |
|---|---|---|---|
| NaCl | 0.5%-5.0% | NaCl | ~ 1.5% |

| Composition C (pH 6.0-7.0) | | Composition F (pH 6.0-7.0) | |
|---|---|---|---|
| Amikacin | 62-78 mg/mL | Amikacin | 65-75 mg/mL |
| Sulfate | | Sulfate | |
| DPPC | 26-38 mg/mL | DPPC | 25-37 mg/mL |
| Cholesterol | 12-20 mg/mL | Cholesterol | 13-20 mg/mL |
| NaCl | 1%-2% | NaCl | 1.2%-1.8% |

It should be noted that increasing amikacin (e.g., amikacin sulfate) concentration alone may not result in a reduced dosing time. For example, in one embodiment, the lipid to amikacin ratio is fixed, and as amikacin concentration is increased (and therefore lipid concentration is increased, since the ratio of the two is fixed, for example at ˜0.7:1 by weight), the viscosity of the solution also increases, which slows nebulization time.

In one embodiment, the method described herein comprises administering to the patient in need of treatment of a MAC lung infection a liposomal amikacin (e.g., amikacin sulfate) composition by inhalation via a nebulizer. The nebulizer provides an aerosol mist of the composition for delivery to the lungs of the patient.

In one embodiment, the nebulizer is selected from an electronic mesh nebulizer, pneumonic (jet) nebulizer, ultrasonic nebulizer, breath-enhanced nebulizer and breath-actuated nebulizer. In one embodiment, the nebulizer is portable. In another embodiment, the nebulizer is one of the nebulizers described in U.S. Patent Application Publication No. 2013/0330400, incorporated by reference herein in its entirety for all purposes.

The principle of operation of a pneumonic nebulizer is generally known to those of ordinary skill in the art and is described, e.g., in Respiratory Care, Vol. 45, No. 6, pp. 609-622 (2000), incorporated by reference herein in its entirety. Briefly, a pressurized gas supply is used as the driving force for liquid atomization in a pneumatic nebulizer. Compressed gas is delivered, which causes a region of negative pressure. The solution to be aerosolized is then delivered into the gas stream and is sheared into a liquid film. This film is unstable and breaks into droplets because of surface tension forces. Smaller particles, i.e., particles with the MMAD and FPF properties described below, can then be formed by placing a baffle in the aerosol stream. In one pneumonic nebulizer embodiment, gas and solution is mixed prior to leaving the exit port (nozzle) and interacting with the baffle. In another embodiment, mixing does not take place until the liquid and gas leave the exit port (nozzle). In one embodiment, the gas is air, O₂ and/or CO₂.

In one embodiment, droplet size and output rate can be tailored in a pneumonic nebulizer. However, consideration should be paid to the composition being nebulized, and whether the properties of the composition (e.g., percentage liposomal complexed amikacin or a pharmaceutically acceptable salt thereof) are altered due to the modification of the nebulizer. For example, in one embodiment, the gas velocity and/or pharmaceutical composition velocity is modified to achieve the output rate and droplet sizes of the present invention. Additionally, or alternatively, the flow rate of the gas and/or solution can be tailored to achieve the droplet size and output rate. For example, an increase in gas velocity, in one embodiment, decreases droplet size. In one embodiment, the ratio of pharmaceutical composition flow to gas flow is tailored to achieve the droplet size and output rate of the invention. In one embodiment, an increase in the ratio of liquid to gas flow increases particle size.

In one embodiment, a pneumonic nebulizer output rate is increased by increasing the fill volume in the liquid reservoir. Without wishing to be bound by theory, the increase in output rate may be due to a reduction of dead volume in the nebulizer. Nebulization time, in one embodiment, is reduced by increasing the flow to power the nebulizer. See, e.g., Clay et al. (1983). Lancet 2, pp. 592-594 and Hess et al. (1996). Chest 110, pp. 498-505.

In one embodiment, a reservoir bag is used to capture aerosol during the nebulization process, and the aerosol is subsequently provided to the patient via inhalation. In another embodiment, the nebulizer provided herein includes a valved open-vent design. In this embodiment, when the patient inhales through the nebulizer, nebulizer output is increased. During the expiratory phase, a one-way valve diverts patient flow away from the nebulizer chamber.

In one embodiment, the nebulizer provided herein is a continuous nebulizer. In other words, refilling the nebulizer with the pharmaceutical composition while administering a dose is not needed. Rather, the nebulizer has at least an 8 mL capacity or at least a 10 mL capacity.

In one embodiment, the nebulizer provided herein does not use an air compressor and therefore does not generate an air flow. In one embodiment, aerosol is produced by the aerosol head which enters the mixing chamber of the device. When the patient inhales, air enters the mixing chamber via one-way inhalation valves in the back of the mixing chamber and carries the aerosol through the mouthpiece to the patient. On exhalation, the patient's breath flows through the one-way exhalation valve on the mouthpiece of the device. In one embodiment, the nebulizer continues to generate aerosol into the mixing chamber which is then drawn in by the patient on the next breath—and this cycle continues until the nebulizer medication reservoir is empty.

In one embodiment, the nebulization time of an effective amount of the pharmaceutical composition is less than about 20 minutes, less than about 18 minutes, less than about 16 minutes or less than about 15 minutes. In one embodiment, the nebulization time of an effective amount of the pharmaceutical composition is less than about 15 minutes. In one embodiment, the nebulization time of an effective amount of the pharmaceutical composition is about 10 minutes to about 14 minutes.

In one embodiment, upon nebulization, about 20% to about 50% by weight of liposomal complexed amikacin or a pharmaceutically acceptable salt thereof, or amikacin or a pharmaceutically acceptable salt thereof encapsulated in a plurality of liposomes, is released from the liposomal complex, due to shear stress on the liposomes. In a further embodiment, the amikacin or pharmaceutically acceptable salt thereof is amikacin sulfate. In another embodiment, upon nebulization, about 25% to about 45% by weight of liposomal complexed or encapsulated amikacin or a pharmaceutically acceptable salt thereof is released from the liposomal complex. In a further embodiment, the amikacin or pharmaceutically acceptable salt thereof is amikacin sulfate. In another embodiment, upon nebulization, about 30% to about 40% by weight of liposomal complexed or encapsulated amikacin or a pharmaceutically acceptable salt thereof is released from the liposomal complex. In a further embodiment, the amikacin or pharmaceutically acceptable salt thereof is amikacin sulfate.

Upon nebulization, in one embodiment, the amount of liposomal complexed amikacin, or a pharmaceutically acceptable salt thereof post-nebulization is about 45% to about 85%, about 50% to about 80%, or about 56% to about 77% of the total weight of the amikacin, or pharmaceutically acceptable salt thereof. These percentages are also referred to herein as “percent associated amikacin, or a pharmaceutically acceptable salt thereof post-nebulization.” In one embodiment, the percent associated amikacin, or a pharmaceutically acceptable salt thereof post-nebulization is from about 50% to about 80% of the total weight of the amikacin, or pharmaceutically acceptable salt thereof. In a further embodiment, the amikacin, or pharmaceutically acceptable salt thereof is amikacin sulfate. In another embodiment, the percent associated amikacin, or a pharmaceutically acceptable salt thereof post-nebulization is about 56% to about 77% of the total weight of the amikacin, or pharmaceutically acceptable salt thereof. In a further embodiment, the amikacin, or pharmaceutically acceptable salt thereof is amikacin sulfate. In one embodiment, the aerosolized composition (i.e., post nebulization) comprises from about 56% to about 77% liposomal complexed amikacin, or a pharmaceutically acceptable salt thereof, and from about 23% to about 44% free amikacin, or a pharmaceutically acceptable salt thereof. In a further embodiment, the amikacin, or pharmaceutically acceptable salt thereof is amikacin sulfate. In one embodiment, the aerosolized composition (i.e., post nebulization) comprises from about 65% to about 75% liposomal complexed amikacin, or a pharmaceutically acceptable salt thereof, and from about 25% to about 35% free amikacin, or a pharmaceutically acceptable salt thereof. In a further embodiment, the amikacin, or pharmaceutically acceptable salt thereof is amikacin sulfate.

In one embodiment, the percent associated amikacin, or a pharmaceutically acceptable salt thereof post-nebulization is measured by reclaiming the aerosol from the air by condensation in a cold-trap, and the liquid is subsequently assayed for free and associated amikacin, or a pharmaceutically acceptable salt thereof. In a further embodiment, the amikacin, or pharmaceutically acceptable salt thereof is amikacin sulfate.

Upon nebulization of the composition described herein, i.e., for administration to a patient in need of treatment of the MAC lung infection, an aerosolized composition is formed, and in one embodiment, the mass median aerodynamic diameter (MMAD) of the aerosolized composition is about 1.0 μm to about 4.2 μm as measured by the Anderson Cascade Impactor (ACI). In one embodiment, the MMAD of the aerosolized composition is about 3.2 μm to about 4.2 μm as measured by the ACI. In one embodiment, the MMAD of the aerosolized composition is about 1.0 μm to about 6 μm as measured by the Next Generation Impactor (NGI). In a further embodiment, the MMAD of the aerosolized composition is about 3 μm to about 5.5 μm as measured by the NGI. In a further embodiment, the MMAD of the aerosolized composition is about 4.1 μm to about 5.3 μm as measured by the NGI.

The fine particle fraction (FPF) of the aerosolized composition, in one embodiment, is greater than or equal to about 64%, as measured by the Anderson Cascade Impactor (ACI), or greater than or equal to about 51%, as measured by the Next Generation Impactor (NGI). In one embodiment, the FPF of the aerosolized composition is greater than or equal to about 70%, as measured by the ACI, greater than or equal to about 51%, as measured by the NGI, or greater than or equal to about 60%, as measured by the NGI.

In one embodiment, the macrolide antibiotic is azithromycin, clarithromycin, erythromycin, carbomycin A, josamycin, kitamycin, midecamycin, oleandomycin, solithromycin, spiramycin, trolcandomycin, tylosin, roxithromycin, or a combination thereof. In a further embodiment, the macrolide antibiotic is administered orally.

In one embodiment, the macrolide antibiotic is azithromycin, clarithromycin, erythromycin, or a combination thereof. In another embodiment, the macrolide antibiotic is clarithromycin. In another embodiment, the macrolide antibiotic is erythromycin. In another embodiment, the macrolide antibiotic is azithromycin. In a further embodiment, the macrolide antibiotic is administered orally. In still a further embodiment, the macrolide antibiotic (e.g., azithromycin) is administered once daily (QD). In one embodiment, ethambutol is administered orally. In a further embodiment, ethambutol is administered once daily (QD).

The pharmaceutical composition comprising amikacin, or a pharmaceutically acceptable salt thereof, the macrolide antibiotic (e.g., azithromycin) and ethambutol may, in one embodiment, be administered QD around the same time each day (e.g., any time of day) in the fasted or as-fed condition.

In one embodiment, about 100 mg to about 350 mg azithromycin is administered orally once-daily. In another embodiment, about 150 mg to about 300 mg azithromycin is administered orally once-daily. In another embodiment, about 200 mg to about 300 mg azithromycin is administered orally once-daily. In another embodiment, about 250 mg azithromycin is administered orally once-daily. In one embodiment, the azithromycin is administered orally as a tablet dosage form.

In one embodiment, about 10 mg/kg to about 25 mg/kg of ethambutol is administered orally once-daily. In another embodiment, about 10 mg/kg to about 20 mg/kg of ethambutol is administered orally once-daily. In another embodiment, about 15 mg/kg of ethambutol is administered orally once-daily. In one embodiment, the ethambutol is administered orally as a tablet dosage form.

In one embodiment, azithromycin 250 mg and ethambutol dosed at 15 mg/kg are taken QD orally, with or without food. In a further embodiment the azithromycin and/or the ethambutol are administered orally in tablet dosage forms.

In one embodiment of the method provided herein, the administration period is from about six months to about twelve months. In another embodiment, the administration period is at least about 12 months. In another embodiment, the administration period is about 12 months. In another embodiment, the administration period is from about 12 to about 36 months. In a further embodiment, the administration period is from about 12 to about 30 months. In even a further embodiment, the administration period is from about 12 to about 24 months.

In the embodiments described herein, the method provides a therapeutic response measured by a PRO generated from one or more PRO instruments, such as Quality of Life Questionnaire-Bronchiectasis (QOL-B), Patient Global Impression of Severity (PGIS) Respiratory, PROMIS Fatigue Short Form 7a (PROMIS F-SF 7a), and Patient Global Impression of Severity (PGIS)-Fatigue. Specifically, the treatment methods provided herein comprise improving a respiratory symptom score as measured by a patient reported outcome (PRO) during the administration period or subsequent to the administration period (e.g., one month after end of the administration period). For example, the respiratory symptom score in one embodiment, comprises the respiratory domain score of the QOL-B or a modified version thereof. In one embodiment, the respiratory symptom score comprises the respiratory domain score of the QOL-B that has been modified to replace the term “bronchiectasis” with “lung condition”. In another embodiment, the respiratory symptom score comprises a subset of the respiratory domain score of the QOL-B or a modified version thereof, i.e., a subset of the nine symptom queries in the QOL-B. In a further embodiment, the respiratory the respiratory domain score of the QOL-B is modified to replace the term “bronchiectasis” with “lung condition”.

The QOL-B is a validated, self-administered, reported outcome questionnaire used to assess symptoms, functioning, and health related quality of life in adults with non-CF bronchicctasis. See, Quittner et al. Quality of Life Questionnaire-Bronchiectasis: final psychometric analyses and determination of minimal important differences scores. Thorax. 2016; 71(1):26-34; incorporated herein by reference in its entirety. It measures outcomes over a recall period of 1 week. The questionnaire contains 37 items on 8 domains (physical, role, vitality, emotional, social, treatment burden, health perception, and respiratory). Each of the 37 items is scored from 1 to 4, and each of the 8 scale scores is standardized on a 0 to 100-point scale, with higher scores representing fewer symptoms or better functioning and quality of life. Scores are calculated for the 7 domains: physical, role, vitality, emotional, social, treatment burden, health perception, and respiratory. For the respiratory domain, a patient is asked on a scale of 1 to 4 (corresponding to “not at all”, “a little”, “a moderate amount” or “a lot”), whether they (i) feel congestion in the chest; (ii) been coughing during the day; (iii) had to cough up mucus. Sputum is also characterized by color: clear, clear to yellow, yellowish-green, brownish-dark, green with traces of blood, or don't know. Patients are also asked on a scale of 1 to 4 (corresponding to “never”, “sometimes”, “often” or “always”), whether they (i) have had shortness of breath with greater of activity; (ii) had wheezing; (iii) had chest pain; (iv) had shortness of breath while talking; (v) woken up during the night because of coughing. Patients are asked to classify their answers to the respiratory domain questions over a recall period of one week.

In some embodiments of the methods of treating MAC lung infections described herein, treating comprises improving the respiratory domain score during the administration period or subsequent to the administration period. It is to be understood that improving the respiratory domain score may comprise the improvement of a subset of respiratory symptoms of the QOL-B, but not every symptom queried.

In one embodiment of the method disclosed herein, the treating comprises improving the respiratory domain score of the QOL-B, for the patient during or subsequent to the administration period, as compared to the respiratory domain score of the patient prior to the treatment (i.e., prior to the administration period). In one embodiment, the QOL-B respiratory domain score for the patient is improved during the administration period. In another embodiment, the respiratory domain score for the patient is improved at the end of the administration period. In still another embodiment, the QOL-B respiratory domain score for the patient is improved about one month after the administration period. In still another embodiment, the QOL-B respiratory domain score for the patient is improved from about one to about three months after the administration period. In still another embodiment, the QOL-B respiratory domain score for the patient is improved about three months after the administration period. In yet another embodiment, the QOL-B respiratory domain score for the patient is improved at least about three months after the administration period. In yet still another embodiment, the QOL-B respiratory domain score for the patient is improved from about three to about six months after the administration period. In another embodiment, the QOL-B respiratory domain score for the patient is improved from about three to about twelve months after the administration period.

In one embodiment of the methods disclosed herein, the treating further comprises improving the score of one or more of the QOL-B non-respiratory domains for the patient during or subsequent to the administration period, as compared to the score of the one or more of the QOL-B non-respiratory domains of the patient prior to the treatment. In a further embodiment, the score of the one or more of the QOL-B non-respiratory domains for the patient is improved during the administration period. In another embodiment, the score of the one or more of the QOL-B non-respiratory domains for the patient is improved at the end of the administration period. In another embodiment, the score of the one or more of the QOL-B non-respiratory domains for the patient is improved about one month after the administration period. In still another embodiment, the score of the one or more of the QOL-B non-respiratory domains for the patient is improved at least about one month after the administration period. In yet another embodiment, the score of the one or more of the QOL-B non-respiratory domains for the patient is improved from about one to about three months after the administration period. In yet still another embodiment, the score of the one or more of the QOL-B non-respiratory domains for the patient is improved about three months after the administration period. In even another embodiment, the score of the one or more of the QOL-B non-respiratory domains for the patient is improved at least about three months after the administration period. In still another embodiment, the score of the one or more of the QOL-B non-respiratory domains for the patient is improved from about three to about six months after the administration period. In yet still another embodiment, the score of the one or more of the QOL-B non-respiratory domains for the patient is improved from about three to about twelve months after the administration period.

In one embodiment of the method disclosed herein, the treating comprises improving one or more respiratory symptoms, as measured by a PGIS Respiratory score, for the patient during or subsequent to the administration period, as compared to the one or more respiratory symptoms of the patient prior to the treatment. The PGIS Respiratory score is a simple categorical rating of symptom severity. The scale is 0=not at all to 5=extremely severe. In one embodiment, the one or more respiratory symptoms as measured by a PGIS Respiratory score, are improved during the administration period. In another embodiment, the one or more respiratory symptoms as measured by a PGIS Respiratory score are improved at the end of the administration period. In another embodiment, the one or more respiratory symptoms for the patient are improved about one month after the administration period. In yet another embodiment, the one or more respiratory symptoms as measured by a PGIS Respiratory score are improved at least about one month after the administration period. In still another embodiment, the one or more respiratory symptoms as measured by a PGIS Respiratory score are improved from about one to about three months after the administration period. In still another embodiment, the one or more respiratory symptoms as measured by a PGIS Respiratory score are improved about three months after the administration period. In yet still another embodiment, the one or more respiratory symptoms as measured by a PGIS Respiratory score are improved at least about three months after the administration period. In even still another embodiment, the one or more respiratory symptoms as measured by a PGIS Respiratory score are improved from about three to about six months after the administration period. In yet even still another embodiment, the one or more respiratory symptoms as measured by a PGIS Respiratory score are improved from about three to about twelve months after the administration period.

The PROMIS F-SF 7a is a self-administered questionnaire assessing a range of self-reported symptoms over the past 7 days, from mild subjective feelings of tiredness to an overwhelming, debilitating, and sustained sense of exhaustion that likely decreases one's ability to execute daily activities and function normalsy in family or social roles. Sec Ameringer et al. Psychometric Evaluation of the Patient-Reported Outcomes Measurement Information System Fatigue-Short Form Across Diverse Populations. Nurs Res. 2016; 65(4):279-289, incorporated herein by reference in its entirety. Fatigue is divided into the experience of fatigue (frequency, duration, and intensity) and the impact of fatigue on physical, mental, and social activities over 7 items. Response options are on a 5-point Likert scale, ranging from 1=never to 5=always. The PROMIS F-SF 7a is universal rather than disease-specific. In one embodiment, the computation of the fatigue score is determined based on results of qualitative work.

In one embodiment of the method disclosed herein, the treating comprises improving one or more fatigue symptoms, as measured by a PROMIS F-SF 7a score, for the patient during or subsequent to the administration period, as compared to the one or more fatigue symptoms of the patient prior to the treatment. In another embodiment, the one or more fatigue symptoms as measured by a PROMIS F-SF 7a score are improved during the administration period. In another embodiment, the one or more fatigue symptoms as measured by a PROMIS F-SF 7a score are improved at the end of the administration period. In still another embodiment, the one or more fatigue symptoms as measured by a PROMIS F-SF 7a score are improved about one month after the administration period. In still another embodiment, the one or more fatigue symptoms as measured by a PROMIS F-SF 7a score are improved at least about one month after the administration period. In yet still another embodiment, the one or more fatigue symptoms as measured by a PROMIS F-SF 7a score are improved from about one to about three months after the administration period. In even another embodiment, the one or more fatigue symptoms as measured by a PROMIS F-SF 7a score are improved about three months after the administration period. In still another embodiment, the one or more fatigue symptoms as measured by a PROMIS F-SF 7a score are improved at least about three months after the administration period. In still another embodiment, the one or more fatigue symptoms as measured by a PROMIS F-SF 7a score are improved from about three to about six months after the administration period. In one embodiment, the one or more fatigue symptoms as measured by a PROMIS F-SF 7a score are improved from about three to about twelve months after the administration period.

The PGIS Fatigue score is a simple categorical rating of symptom severity. The scale is 0=not at all to 5=extremely severe. In one embodiment of the method disclosed herein, the treating comprises improving one or more fatigue symptoms, as measured by a PGIS Fatigue score, for the patient during or subsequent to the administration period, as compared to the one or more fatigue symptoms of the patient prior to the treatment. In one embodiment, the one or more fatigue symptoms as measured by a PGIS Fatigue score are improved during the administration period. In another embodiment, the one or more fatigue symptoms as measured by a PGIS Fatigue score are improved at the end of the administration period. In another embodiment, the one or more fatigue symptoms as measured by a PGIS Fatigue score are improved about one month after the administration period. In still another embodiment, the one or more fatigue symptoms as measured by a PGIS Fatigue score are improved at least about one month after the administration period. In still another embodiment, the one or more fatigue symptoms as measured by a PGIS Fatigue score are improved from about one to about three months after the administration period. In yet another embodiment, the one or more fatigue symptoms as measured by a PGIS Fatigue score are improved about three months after the administration period. In yet still another embodiment, the one or more fatigue symptoms as measured by a PGIS Fatigue score are improved at least about three months after the administration period. In yet another embodiment, the one or more fatigue symptoms as measured by a PGIS Fatigue score are improved from about three to about six months after the administration period. In even another embodiment, the one or more fatigue symptoms as measured by a PGIS Fatigue score are improved from about three to about twelve months after the administration period.

In some embodiments, the therapeutic response resulting from the method provided herein is measured by microbiological assessment of sputum specimens from the patient, e.g., achieving a negative MAC sputum culture, or a MAC sputum culture conversion, defined herein as two consecutive negative MAC sputum cultures, the rate of recurrence of MAC, and the rate of developing a MAC isolate with amikacin minimum inhibitory concentration (MIC)≥128 μg/mL.

In one embodiment of the method disclosed herein, the treating comprises achieving a negative MAC sputum culture in the patient during or subsequent to the administration period.

In one embodiment of the method disclosed herein, the treating comprises decreasing the length of time to achieve a first negative MAC sputum culture as compared to a patient with a newly diagnosed, untreated MAC lung infection administered the macrolide antibiotic and ethambutol, but not the pharmaceutical composition, for the same administration period.

In one embodiment of the method disclosed herein, the treating comprises achieving a MAC sputum culture conversion in the patient during or subsequent to the administration period. In a further embodiment, the patient achieves the MAC sputum culture conversion during the administration period. In still another embodiment, the patient achieves the MAC sputum culture conversion at the end of the administration period. In still another embodiment, the patient achieves the MAC sputum culture conversion about one month after the administration period. In still another embodiment, the patient achieves the MAC sputum culture conversion at least about one month after the administration period. In yet another embodiment, the patient achieves the MAC sputum culture conversion from about one to about three months after the administration period. In even another embodiment, the patient achieves the MAC sputum culture conversion about three months after the administration period. In still another embodiment, the patient achieves the MAC sputum culture conversion at least about three months after the administration period. In even another embodiment, the patient achieves the MAC sputum culture conversion from about three to about six months after the administration period. In still another embodiment, the patient achieves the MAC sputum culture conversion from about three to about twelve months after the administration period.

In another embodiment, the treating comprises achieving a durable culture conversion in the patient. In embodiments described herein, durable culture conversion refers to the continued presence of a negative MAC sputum culture after culture conversion is achieved. For example, in one embodiment, durable culture conversion refers to a patient having a negative MAC sputum culture about 1 month after culture conversion, at about 1 month and about 3 months after culture conversion, at about 1 month and about 6 months after culture conversion, at about 1 month and about 9 months after culture conversion or at about 1 month and about 12 months after culture conversion.

In one embodiment of the method disclosed herein, the treating comprises decreasing the length of time to achieve a MAC sputum culture conversion as compared to a patient with a newly diagnosed, untreated MAC lung infection administered the macrolide antibiotic and ethambutol, but not the pharmaceutical composition, for the same administration period.

In one embodiment of the method disclosed herein, the treating comprises reducing the rate of recurrence of MAC for the patient, as compared to the rate of recurrence of MAC for a patient with a newly diagnosed, untreated MAC lung infection administered the macrolide antibiotic and ethambutol, but not the pharmaceutical composition, for the same administration period. In one embodiment, the recurring MAC is of the same species and genome as the MAC prior to the treatment. In another embodiment, the recurring MAC is of a different species, or of the same species but a different genome, as compared to the MAC prior to the treatment.

In one embodiment of the method disclosed herein, the treating comprises decreasing the rate of developing a MAC isolate with amikacin minimum inhibitory concentration (MIC)≥128 μg/mL for the patient, as compared to the rate of developing a MAC isolate with amikacin MIC≥128 μg/mL for a patient with a newly diagnosed, untreated MAC lung infection administered the macrolide antibiotic and ethambutol, but not the pharmaceutical composition, for the same administration period.

In some embodiments, the therapeutic response resulting from the method provided herein is measured by actigraphy, e.g., daily activity and sleep efficiency using an actigraphy device.

In one embodiment of the method disclosed herein, the treating comprises improving the daily activity and sleep efficiency, as measured by a Philips ACTIWATCH SPECTRUM PRO actigraphy device, for the patient during or subsequent to the administration period, as compared to the daily activity and sleep efficiency experienced by the patient prior to the treatment, or as compared to the daily activity and sleep efficiency of a patient with a newly diagnosed, untreated MAC lung infection administered the macrolide antibiotic and ethambutol, but not the pharmaceutical composition, for the same administration period. In a further embodiment, the daily activity and sleep efficiency for the patient is improved during the administration period. In still a further embodiment, the daily activity and sleep efficiency for the patient is improved at the end of the administration period. In still a further embodiment, the daily activity and sleep efficiency for the patient is improved about one month after the administration period. In still a further embodiment, the daily activity and sleep efficiency for the patient is improved at least about one month after the administration period. In still a further embodiment, the daily activity and sleep efficiency for the patient is improved from about one to about three months after the administration period. In still a further embodiment, the daily activity and sleep efficiency for the patient is improved about three months after the administration period. In still a further embodiment, the daily activity and sleep efficiency for the patient is improved at least about three months after the administration period. In still a further embodiment, the daily activity and sleep efficiency for the patient is improved from about three to about six months after the administration period. In still a further embodiment, the daily activity and sleep efficiency for the patient is improved from about three to about twelve months after the administration period.

In one embodiment, the method provided herein further comprises administering the patient a rifamycin compound. In a further embodiment, the rifamycin compound is rifampin, rifabutin, rifapentine, rifaximin, or a combination thereof. In still a further embodiment, the rifamycin compound is rifampin.

In one embodiment, the method herein excludes administering to the patient a rifamycin compound.

### EXAMPLES

The present invention is further illustrated by reference to the following Examples. However, it should be noted that the Examples, like the embodiments described above, are illustrative and are not to be construed as restricting the scope of the invention in any way.

### Example 1—Validation of Patient-Reported Outcome Instruments in Adult Subjects with Newly Diagnosed Nontuberculous Mycobacterial (NTM) Lung Infection Caused by Mycobacterium avium Complex (MAC)

There remains an unmet medical need for patients with non-cavitary lung disease and newly diagnosed (initial or subsequent) MAC lung infections, who have not started treatment. There is currently no validated patient reported outcome (PRO) instrument to evaluate symptoms in patients with MAC lung disease. This example describes a randomized, double-blind, placebo-controlled, active comparator, multicenter study to validate PRO instruments in adult subjects with non-cavitary lung disease and newly diagnosed (initial or subsequent) MAC lung infections, who have not started treatment. This study will assess data generated from PRO instruments, with an aim to generate evidence demonstrating the domain specification (via modern psychometric methods), reliability, validity, and responsiveness (within-subject meaningful change) of the PRO endpoints of amikacin liposome inhalation suspension (ALIS)-based regimen within the MAC lung disease population, e.g., to validate PRO instruments of QOL-B and PROMIS F-SF 7a within the MAC lung disease population. The instruments validated in this study will be used for the assessment of the clinical benefit in a separate study described in Example 2.

The ALIS used in the study is an approved ARIKAYCE® drug product under NDA 207356 (ARIKAYCE®). The ALIS is a sustained-release lipid composition of amikacin indicated in adults who have limited or no alternative treatment options, for the treatment of Mycobacterium avium complex (MAC) lung disease as part of a combination antibacterial drug regimen in patients who do not achieve negative sputum cultures after a minimum of 6 consecutive months of a multidrug background regimen therapy.

A matching placebo referred to as empty liposome control (ELC) for the ALIS uses the same excipients as the ALIS in the absence of amikacin drug substance. The formulation composition comparison of the ALIS and its matching placebo (ELC) is shown in Table 2.

TABLE 2
ALIS and ELC Compositions

| Component | ALIS | | ELC |
|---|---|---|---|
| Amikacin Sulfate | 70 | mg/mL | N/A |
| DPPC | About 32 | mg/mL | 1.5 mg/mL |
| Cholesterol | About 16 | mg/mL | 0.75 mg/mL |

| | | |
|---|---|---|
| NaCl | 1.5% (w/w) | 1.5% (w/w) |

The manufacturing process of the diluted ELC uses the similar liposome formation processing process and the same equipment that is used for ALIS. The manufacturing process for the ELC has been developed to produce liposomes that are comparable to ALIS in which the amikacin sulfate is replaced with a 1.9% sodium chloride during infusion and liposome formation. Once the liposomes are formed, the defiltration is started using 1.5% sodium chloride to the targeted lipid concentrations, e.g., 5% ELC.

The two comparators, azithromycin and ethambutol tablets, will be obtained from commercial sources.

### Methods

### Subject Eligibility Criteria

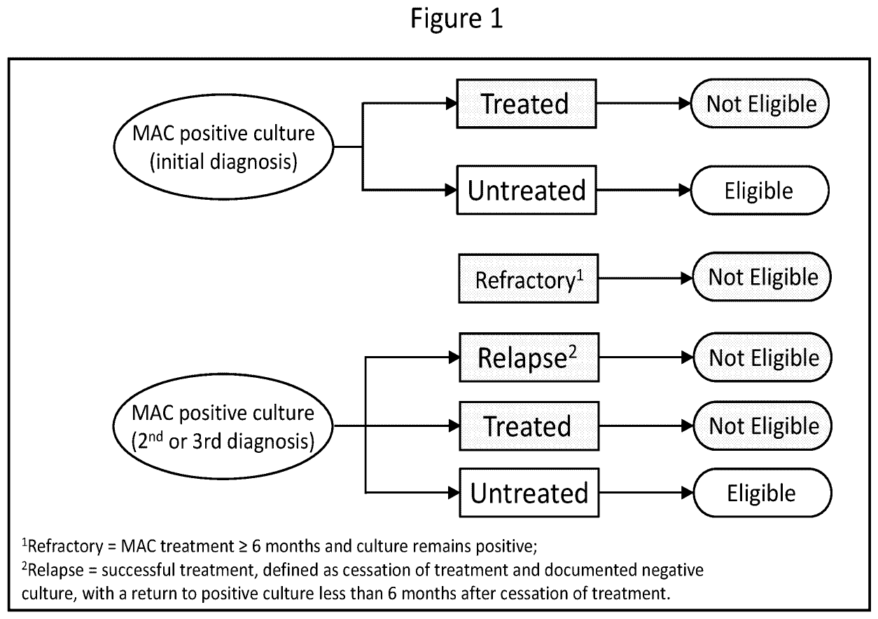

The patient population under investigation for this clinical study will include adults with non-cavitary lung disease and a new diagnosis (initial or subsequent) MAC lung infection who have not started treatment (FIG. 1). Table 3 below provides the inclusion criteria for the study.

TABLE 3

| | Inclusion criteria for the study |
|---|---|
| □ | Male or female, ≥18 years of age |
| □ | Current diagnosis of MAC lung infection. MAC or mixed |
| | infection with MAC as the dominant species allowed, with |
| | MAC as the intended organism for treatment |
| □ | Positive sputum culture for MAC within 6 months prior to |
| | Screening |
| □ | Positive sputum culture for MAC at Screening |
| □ | A high-resolution chest computed tomography (CT) scan or chest |
| | CT scan with contrast, read locally, within 6 months prior to |
| | Screening. Subjects who do not have a chest CT scan within |
| | 6 months prior to Screening will be required to obtain a high- |
| | resolution chest CT scan or chest CT scan with contrast, read |
| | locally, during Screening. |
| □ | In the Investigator's opinion, documented respiratory signs/ |
| | symptoms at Screening that are attributable to the current MAC |
| | lung infection |
| □ | An average QOL-B respiratory domain score of ≤85 based on |
| | scores at Screening and on the day of enrollment prior to |
| | randomization |
| □ | In the Investigator's opinion, underlying lung disease (e.g., chronic |
| | obstructive pulmonary disease (COPD), bronchiectasis) have |
| | been managed according to best local standard of care, and on |
| | stable maintenance therapy for a minimum of 4 weeks prior |
| | to randomization |

Table 4 below provides certain exclusion criteria for the study.

TABLE 4

| | Exclusion criteria for the study |
|---|---|
| □ | History of more than three (3) prior MAC lung infections |
| □ | Received any mycobacterial antibiotic treatment for current |
| | MAC lung infection |
| □ | Refractory MAC lung infection, defined as having positive MAC |
| | cultures while being treated with a multi drug mycobacterial |
| | antibiotic treatment regimen for a minimum of 6 consecutive |
| | months and no documented successful treatment, defined as |
| | negative sputum culture for MAC and cessation of treatment |
| □ | Relapse of prior MAC lung infection, defined as positive sputum |
| | culture for MAC ≤ 6 months of cessation of prior successful |
| | treatment |
| □ | MAC isolate with MIC for amikacin ≥ 128 μg/mL at Screening |
| □ | Evidence of any pulmonary cavity ≥ 2 cm in diameter, as |
| | determined by chest CT scan, read locally, within 6 months |
| | prior to Screening |
| □ | Radiographic finding of new lobar consolidation, atelectasis, |
| | significant pleural effusion, or pneumothorax during routine |
| | clinical care within 2 months prior to Screening |
| □ | Active pulmonary malignancy (primary or metastatic) or any |
| | malignancy requiring chemotherapy or radiation therapy within |
| | 1 year prior to Screening or anticipated during the study |
| □ | Active pulmonary tuberculosis requiring treatment during |
| | Screening |
| □ | Hospitalization for underlying lung disease at Screening |
| □ | Acute pulmonary exacerbation (e.g., COPD or bronchiectasis) |
| | requiring treatment with antibiotics, or corticosteroids |
| | (intravenous (IV) or oral), within 4 weeks prior to and |
| | during Screening |
| □ | Predicted forced expiratory volume in 1 second (FEV₁) < 35%, |
| | pre-bronchodilator use |
| □ | Use of inhaled or systemic aminoglycosides with activity against |
| | MAC (e.g., amikacin, kanamycin, or streptomycin) during |
| | Screening |
| □ | Prior exposure to ALIS (including clinical study) |
| □ | Positive pregnancy test or lactation at Screening. |
| □ | Administration of any investigational drug within 8 weeks |
| | prior to Screening. |

### Study Design

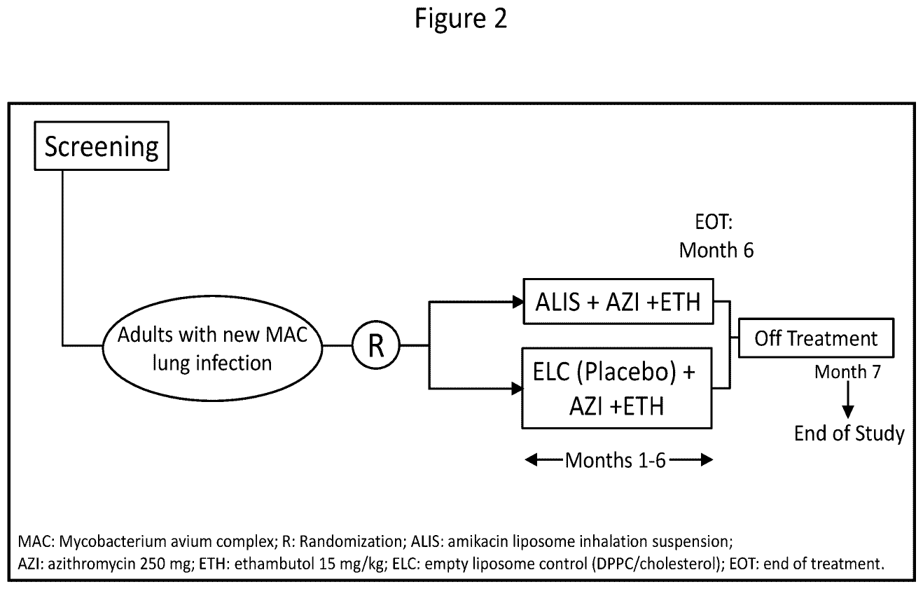

FIG. 2 is a schematic diagram of the study design and treatment duration. The study will be conducted in a double-blinded fashion, including blinding to treatment group assignments and microbiology results, allowing for an accurate assessment of the efficacy and safety of an ALIS-based regimen. The study also includes an active comparator, which allows for the determination of the incremental benefit of ALIS and is an appropriate, efficient, and accurate control for the study population.

Eligible subjects will be randomized at Baseline in a 1:1 ratio to receive one of the two treatment regimens: ALIS+azithromycin (AZI)+ethambutol (ETH) or empty liposome control (ELC)+AZI+ETH for 6 months, and then remain off all anti-MAC treatments for 1 month, with a final EOS evaluation at Month 7. ALIS 590 mg or ELC will be administered once daily (QD) by inhalation via nebulization over approximately 6 minutes to up to 15 minutes. The nebulizer system is manufactured by PARI Respiratory Equipment, Richmond, VA, a subsidiary of PARI Pharma GmbH, Germany. Azithromycin 250 mg tablets and ethambutol 15 mg/kg tablets will be taken QD by mouth, with or without food. Screening period will be up to approximately −70 days to −28 days (about 2.5 months). The duration of the study will be up to 9.5 months from the Screening visit to EOS. The EOS is defined as the date of the last visit of the last subject.

Randomization will be stratified by region and history of MAC lung infection (initial or subsequent). After Baseline, subjects will return to the study site for in-clinic visits at Months 1, 3, 5, 6/end of treatment (EOT), and 7/end of study (EOS). On the day of study visits, study drug will be administered at the study site by study personnel after collection of PRO instruments and after sputum collection.

Visits at Months 2 and 4 do not require in-clinic appointments. At these non-in clinic visits, AEs and concomitant medications will be assessed and subjects will be required to produce and ship sputum samples. At the Month 6/EOT visit, subjects will discontinue all study treatments and will be followed for a 1-month off treatment period, during which initiation of any new medical or non-medical therapies for MAC lung infection should be avoided.

At Month 7/EOS, subjects will complete all protocol-specified assessments and EOS procedures.

The PROs to be validated within the MAC lung disease population are provided in Table 5:

TABLE 5
PROs

| | |
|---|---|
| 1. Quality of Life- | Subjects will self-administer the QOL-B |
| Bronchiectasis | questionnaire electronically. The QOL-B |
| (QOL-B) | questionnaire will be the first assessment to |
| | be conducted at the specified study visits. |
| 2. PROMIS Fatigue | Subjects will self-administer and complete the |
| Short Form (FSF) 7a | PROMIS F-SF 7a questionnaire electronically |
| (PROMIS F-SF 7a) | at study visits. The PROMIS F-SF 7a will be |
| | the second assessment to be conducted at the |
| | specified study visits. |

Patient-reported validating variables are provided in Table 6.

TABLE 6
Patient-reported validating variables.

| | |
|---|---|
| 1. | The EXACT PRO was developed and qualified for use in characterizing |
| EXAcerbations | COPD clinical endpoints. See Jones et al., EXACT-PRO Study Group, |
| of Chronic | Characterizing and quantifying the symptomatic features of COPD |
| Pulmonary | exacerbations. Chest. 2011; 139(6): 1388-94; Leidy et al. Development |
| Disease Tool | of the EXAcerbations of Chronic Obstructive Pulmonary Disease Tool |
| (EXACT) | (EXACT): a patient-reported outcome (PRO) measure. Value Health. |
| | 2010; 13(8): 965-75; Leidy et al. Standardizing measurement of chronic |
| | obstructive pulmonary disease exacerbations. Reliability and validity of |
| | a patient-reported diary. Am J Respir Crit Care Med. 2011; 183(3): 323- |
| | 9; United States Food and Drug Administration (FDA). COA DDT |
| | 0003: Exacerbations of Chronic Pulmonary Disease Tool (EXACT). |
| | Center for Drug Evaluation and Research; 2013; each of which is |
| | incorporated herein by reference in its entirety. |
| | Several of the concepts assessed by the EXACT are relevant to |
| | characterizing the symptomatic phenomena expected within |
| | bronchiectasis. These include, but are not limited to, dyspnea and |
| | fatigue. The EXACT total score measures acute, sustained, and |
| | worsening signs and symptoms exceeding subject-specific expected |
| | variability. The EXACT PRO is composed of 14 items from which a |
| | total score is generated. Domains assessed by the EXACT PRO include |
| | dyspnea, cough and sputum production, chest symptoms, difficulty |
| | expectorating, fatigue, sleep disturbance, and fear or concern. |
| | Subjects will self-administer and complete the EXACT PRO |
| | questionnaire electronically at study visits. |
| 2. EXACT | The EXACT-RS is an 11-item subset of the EXACT PRO assessing |
| Respiratory | exacerbations of respiratory symptoms. See Leidy et al. Measuring |
| Symptoms | respiratory symptoms of COPD: performance of the EXACT- |
| (EXACT-RS) | Respiratory Symptoms Tool (E-RS) in three clinical trials. Respir Res. |
| | 2014; 15: 124; Leidy et al. Measuring respiratory symptoms in clinical |
| | trials of COPD: reliability and validity of a daily diary. Thorax. |
| | 2014; 69(5): 443-9; each of which is incorporated herein by reference in |
| | its entirety. |
| | Subjects will self-administer and complete the EXACT-RS |
| | questionnaire electronically at study visits. |
| 3. St. George | The SGRQ is a self-administered instrument for the assessment of |
| Respiratory | overall health, daily life, and perceived well-being among individuals |
| Questionnaire | with obstructive airways disease. See Jones et al. A self-complete |
| (SGRQ) | measure of health status for chronic airflow limitation. The St. George's |
| | Respiratory Questionnaire. Am Rev Respir Dis. 1992; 145(6): 1321-7, |
| | incorporated herein by reference in its entirety. |
| | The instrument consists of 50 items with 76 weighted responses |
| | grouped into a set of 17 questions. The items are divided into 2 parts |
| | and 3 categories: symptom, activity, and impact. Part 1 (symptom |
| | component) assesses an individuals' perception of their recent |
| | respiratory problems. Part 1 evaluates frequency and severity of |
| | symptoms including cough, sputum production, wheezing, |
| | breathlessness, and the duration and frequency of attacks of |
| | breathlessness and wheeze. Part 2 (activity and impact components) |
| | addresses individuals' current state. The activity component is |
| | composed of a series of items evaluating metabolic equivalence of |
| | incrementally increasingly physically demanding activities, ranging |
| | from sitting to climbing a flight of stairs. The impact component |
| | measures domains of social functioning and psychological disturbances |
| | resulting from airway disease. |
| | Subjects will self-administer and complete the SGRQ questionnaire |
| | electronically at study visits. |
| 4. Functional | The FACIT-Fatigue Scale is a short, 13-item, easy to administer, tool |
| Assessment of | measuring an individual's level of fatigue during their usual daily |
| Chronic Illness | activities during the past week. See Smith et al. Building a measure of |
| Therapy | fatigue: the functional assessment of Chronic Illness Therapy Fatigue |
| (FACIT) - | Scale. PM R. 2010; 2(5): 359-63, incorporated herein by reference in its |
| Fatigue Scale | entirety. |
| | The level of fatigue is measured on a four-point Likert scale (4 = not at |
| | all fatigued to 0 = very much fatigued). FACIT-Fatigue is routinely |
| | administered in studies of respiratory disease populations for accurately |
| | evaluating the common sequela of fatigue. |
| | The FACIT-Fatigue will be completed at study visits. Subjects will self- |
| | administer the questionnaire electronically. |
| 5. PGIS | The PGIS Respiratory will be completed at study visits. Subjects will |
| Respiratory | self-administer the questionnaire electronically. |
| 6. PGIS- | The PGIS Fatigue will be completed at study visits. Subjects will self- |
| Fatigue | administer the questionnaire electronically. |

Clinical/digital validating variables include:
1. Percent predicted forced expiratory volume (ppFEV₁)
2. Sputum culture

Microbiological assessment of sputum specimens will be used for secondary efficacy measurements.

During the study, pre-dose expectorated or induced sputum samples (approximately 2 mL) are required at study visits. To improve the probability of obtaining a good sputum specimen, 2 sputum samples will be obtained from each subject at each assessment; subjects will produce 1 sputum sample on the day prior to the scheduled visit and bring it to the scheduled visit, and 1 sputum sample on the day of the visit prior to dosing with study drugs. At Screening, subjects will produce 1 sputum sample on the day of the visit and 1 sputum during the week following the Screening visit. If a subject is unable to produce sputum spontaneously, sputum may be induced. If after induction, a subject is still unable to produce sputum despite reasonable efforts, this will be recorded as non-productive at that time point. At the scheduled visit, once sputum is collected (spontaneously or by induction), study drugs can be administered.

For microbiological assessment, sputum specimens will be cultured in broth media (liquid) in addition to agar media (solid), and will be held for up to 6 weeks. A negative culture result will not be reported until after this time has transpired.

Growth of other bacteria due to co-infections (e.g., Pseudomonas aeruginosa, M. abscessus, other NTM) will be reported. Colony counts of MAC growth will be reported. Isolates of MAC will be identified using a commercial RNA probe (AccuProbe, Gen Probe, Inc., GenoType NTM-DR Ver 1.0, Hain Lifescience) and subsequently identified to species using molecular methodology. See Cousins et al. Multiplex PCR provides a low-cost alternative to DNA probe methods for rapid identification of Mycobacterium avium and Mycobacterium intracellulare. J Clin Microbiol. 1996; 34(9):2331-2333, incorporated herein by reference in its entirety. Standard antibiotic sensitivity testing using MICs will be routinely performed on mycobacterial isolates. See CLSI. Performance Standards for Susceptibility Testing of Mycobacteria, Nocardia spp, and Other Aerobic Actinomycetes. 1st ed. CLSI standard M62. Wayne PA: Clinical Laboratory Standards Institute; 2018; CLSI. Susceptibility Testing of Mycobacteria, Nocardia spp, and Other Aerobic Actinomycetes. 3rd ed. CLSI standard M24. Wayne PA: Clinical Laboratory Standards Institute; 2018; Forbes et al. M48-A. Laboratory Detection and Identification of Mycobacteria; Approved Guideline. Clinical and Laboratory Standard Institute. 2008; 28(17); each of which is incorporated herein by reference in its entirety.

The MAC species and genotype at the Screening and/or Baseline sputum specimen will be identified by whole genome sequencing (Illumina Nextseq500™). In subjects who achieve sputum culture conversion and who subsequently have positive sputum culture(s) (broth or agar), typing by whole genome sequencing will also be conducted.

MAC culture assessment definitions of this study are listed in Table 7.

TABLE 7
MAC culture assessment definitions

| | |
|---|---|
| MAC culture negative | No MAC growth on agar media and broth media in all sputum |
| | cultures at a visit. |
| Non-productive | Prior to culture conversion: visits where subjects were recorded |
| | as non-productive are considered MAC culture positive. |
| | Post culture conversion: visits where subjects were recorded as |
| | non-productive are considered MAC culture negative, since |
| | subject has demonstrated consistent conversion of cultures to |
| | negative. |
| Culture conversion | No MAC growth on agar media and broth media in all sputum |
| | cultures at 2 consecutive visits. |
| Recurrence: | Subsequent to culture conversion, a MAC positive culture that is |
| Relapse | the same species and genome as cultured at Screening/Baseline. |
| | The positive culture is defined as one positive culture in agar |
| | media or positive cultures in broth media at 2 consecutive visits. |
| Recurrence: New | Subsequent to culture conversion, a MAC positive culture that is |
| MAC infection | different than cultured at Screening/Baseline (different species, or |
| | same specie but different genome). The positive culture is defined |
| | as one positive culture in agar media or positive cultures in broth |
| | media at 2 consecutive visits. |

3. Assessment of MAC isolated with amikacin MIC≥128 μg/mL
4. Actigraphy

The Philips ACTIWATCH SPECTRUM PRO actigraphy device resembles a wristwatch and senses the activity of the wearer. It is to be worn continuously by the subject throughout the duration of the study. The data collected is downloaded at each in-clinic visit or during a home care visit.

### Outcomes Assessment

Some of the objectives and endpoints of the study are shown in Table 8.

TABLE 8

| Primary Objective | Primary Endpoint | |
|---|---|---|
| To generate evidence | Findings on psychometric validation optimized | |
| demonstrating the domain | and reported for: | |
| specification (via modern | 1) | Cross-sectional validation (modem |
| psychometric methods), reliability, | | psychometrics, internal consistency, |
| validity, and responsiveness | | concurrent validity, and known- groups |
| (within-subject meaningful | | validity) at Baseline. |
| change) of the patient-reported | 2) | Test-retest reliability between Screening and |
| outcome (PRO) endpoints | | Baseline among subjects reporting no change |
| | | on Patient Global Impression of Severity |
| | | (PGIS) between Screening and Baseline. |
| | | PGIS anchors will be PRO specific, with a |
| | | respiratory and fatigue PGIS applied to the |
| | | Quality of Life - Bronchiectasis (QOL-B) |
| | | respiratory domain and Patient-Reported |
| | | Outcome Measurement Information System - |
| | | Fatigue-Short Form 7a (PROMIS F-SF 7a), |
| | | respectively. |
| | 3) | Within-subject meaningful change estimated |
| | | via anchor-based methods and validated via |
| | | empirical cumulative distribution functions |
| | | (eCDFs) and probability density functions |
| | | (ePDFs) between Baseline and End of Study |
| | | (EOS) (Month 7). |

| Secondary Objectives: | Secondary Endpoints |
|---|---|
| To evaluate the effect of each | |
| treatment arm (amikacin liposome | |
| inhalation suspension [ALIS] + | |
| azithromycin [AZI] + ethambutol | |
| [ETH] and empty liposome control | |
| [ELC] + | |
| AZI + ETH) on the following: | |
| 1. Culture conversion by end of | Proportion of subjects achieving culture conversion |
| treatment (EOT) (Month 6) | by EOT (Month 6) (negative cultures for MAC at |
| | Month 5 and Month 6) |
| 2. Patient-reported respiratory | Change from Baseline to 1 month off treatment |
| symptoms at 1 month off | (Month 7) in respiratory symptoms (as assessed by |
| treatment (Month 7) | QOL-B) |

### Statistical Analysis

Cross-sectional validation will be conducted at Baseline and will consist of modern psychometric methods (exploratory factor analysis, item response theory models and corresponding assessments of local dependence and differential item functioning), internal consistency, concurrent validity, and known groups validity.

Test-retest reliability between Screening and Baseline among subjects reporting no change on PGIS between Screening and Baseline. PGIS anchors will be PRO specific, with a respiratory and fatigue PGIS applied to the QOL-B respiratory domain and PROMIS F-SF 7a, respectively.

Within-subject meaningful change estimated via anchor-based methods and validated via eCDFs and ePDFs between Baseline and EOS (Month 7). Anchors employed will include the PGIS and culture conversion.

All of the summaries pertaining to secondary endpoints will utilize an intent-to-treat (ITT) analysis set on available data, unless otherwise stated in Statistical Analysis Plan (SAP).

Reporting of categorial secondary endpoints will include basic statistics, estimates derived via logistic regression and will include estimates of rates, rate differences, and odds ratios together with corresponding 95% confidence intervals (CIs) resulting, as appropriate.

Reporting of continuous secondary endpoints will include basic statistics, estimates derived from analysis of covariance (ANCOVA) model with change from Baseline as response variable and treatment and Baseline as dependent variables regression and will include estimates with corresponding 95% CIs as appropriate.

Analysis of variance and logistic regression models may not include adjustment for randomization strata due to expected small counts within each combination of strata.

Time to culture conversion will be summarized via Kaplan-Meier method.

The analysis of safety data will be conducted on the safety analysis set (all randomly assigned subjects who receive at least 1 dose of ALIS, ELC, AZI, or ETH). Safety parameters for each treatment group will include the occurrence of adverse events (AEs), the use of concomitant medications, and changes in clinical laboratory values (serum chemistry, hematology, and urinalysis), vital signs measurements, and physical examination findings (including body weight) between Baseline and EOT. Adverse events will be coded according to the latest version of the Medical Dictionary for Regulatory Activities (MedDRA) dictionary. Summaries will be presented for all AEs, AEs determined by the Investigator to be treatment-related, serious adverse events (SAEs), and AEs causing withdrawal from the study. Hematology, chemistry, and urinalysis values will be summarized by treatment group over time and by visit.

### Example 2—Evaluation of the Efficacy and Safety of an Amikacin Liposome Inhalation Suspension (ALIS)-Based Regimen in Adult Subjects with Newly Diagnosed Nontuberculous Mycobacterial (NTM) Lung Infection Caused by Mycobacterium avium Complex (MAC)

This example describes a randomized, double-blind, placebo-controlled, active comparator, multicenter study to evaluate the efficacy and safety of the amikacin liposome inhalation suspension (ALIS)-based regimen, as described in Example 1, in adult subjects with non-cavitary lung disease and newly diagnosed (initial or subsequent) MAC lung infections, who have not started treatment, also as described in Example 1, using the PRO instruments, e.g., QOL-B and PROMIS F-SF 7a, validated in Example 1. This study also aims to evaluate the efficacy and safety of the ALIS-based regimen in the rate of durable sputum culture conversion off-treatment in the study population.

### Methods

### Subject Eligibility Criteria

The patient population under investigation for this clinical study will include adults with non-cavitary lung disease and a new diagnosis (initial or subsequent) MAC lung infection who have not started treatment. The subject eligibility criteria are the same as those for the study of Example 1, shown in FIG. 1 and Tables 3 and 4.

### Study Design

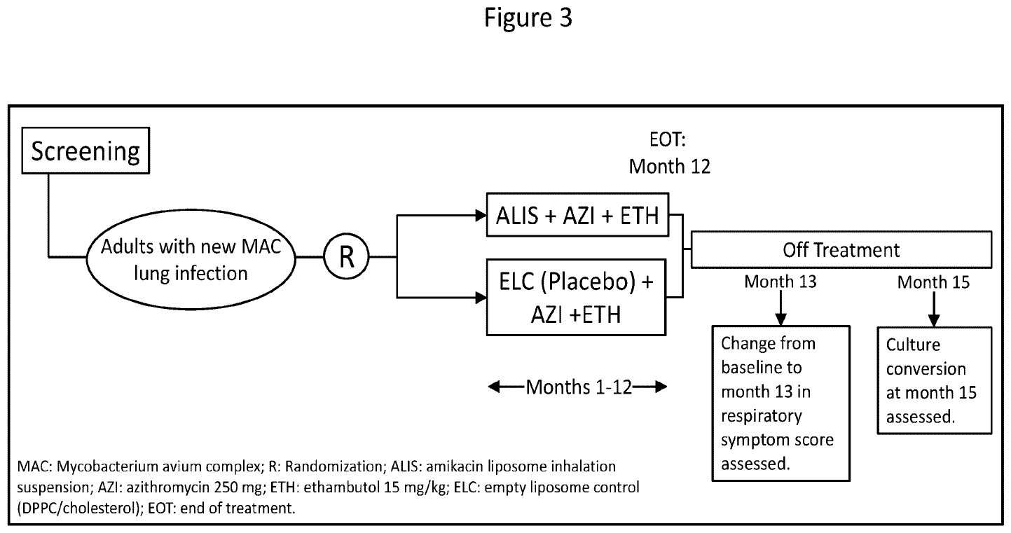

FIG. 3 is a schematic diagram of the study design and treatment duration. Similar to the study of Example 1, this study will be conducted in a double-blinded fashion, including blinding to treatment group assignments and microbiology results, and includes an active comparator.

Eligible subjects with a new diagnosis (initial or subsequent) of MAC lung infection who have not started treatment will be randomized at Baseline in a 1:1 ratio to receive one of the two treatment regimens: ALIS+azithromycin (AZI)+ethambutol (ETH) or empty liposome control (ELC)+AZI+ETH for 12 months, and then remain off all MAC treatments for 3 months, with a final EOS evaluation at Month 15. The compositions of ALIS and ELC are as described in Table 2 of Example 1. ALIS 590 mg or ELC will be administered once daily (QD) by inhalation via nebulization over approximately 6 minutes to up to 15 minutes. The nebulizer system is manufactured by PARI Respiratory Equipment, Richmond, VA, a subsidiary of PARI Pharma GmbH, Germany. Azithromycin 250 mg tablets and ethambutol 15 mg/kg tablets will be taken QD by mouth, with or without food. The Screening period will be up to approximately −70 days to −28 days (approximately 2.5 months). The duration of the study for each subject will be up to 17.5 months from the Screening visit to EOS. The EOS is defined as the date of the last visit of the last subject.

Randomization will be stratified by region and history of MAC lung infection (initial or subsequent). After Baseline, subjects will return to the study site for in-clinic visits at Months 1, 3, 5, 6, 9, 11, 12/EOT, 13, and 15/EOS. EOT: end of treatment; EOS: end of study.

Visits at Months 2, 4, 7, 8, and 10 do not require in-clinic appointments. At these non-in clinic visits, adverse events (AEs) and concomitant medications will be assessed, eDiary data will be collected for assessment of study drug intake, and subjects will be required to provide and ship sputum samples.

At the Month 12/EOT visit, subjects will discontinue all study treatments and will be followed for a 3-month off-treatment period, during which medical or non-medical therapies for MAC lung infection should not be given.

At Month 13, subjects will complete all protocol-specified assessments. At Month 15/EOS, subjects will complete all protocol-specified assessments and the final EOS procedures.

Subjects will complete the following PRO assessments.

### 1. QOL-B

The QOL-B questionnaire will be completed at study visits. Subjects will self-administer the questionnaire electronically. The QOL-B questionnaire will be the first assessment to be conducted at the specified study visits. The computation of the respiratory score will be derived from selected items within the QOL-B instrument that will be determined based on results of qualitative work.

### 2. PROMIS F-SF 7a

The PROMIS F-SF 7a will be completed at study visits. Subjects will self-administer the questionnaire electronically. The PROMIS F-SF 7a will be the second assessment to be conducted at the specified study visits. The computation of the fatigue score will be determined based on results of qualitative work.

### 3. PGIS-Respiratory

The PGIS Respiratory will be completed at study visits. Subjects will self-administer the questionnaire electronically.

### 4. PGIS-Fatigue

The PGIS Fatigue will be completed at study visits. Subjects will self-administer the questionnaire electronically.

### Outcomes Assessment

Certain objectives and endpoints of the study are shown in Table 9.

TABLE 9

| Primary Objective | Primary Endpoint |
|---|---|
| To evaluate the efficacy of ALIS + | Change from Baseline to |
| azithromycin (AZI) + ethambutol (ETH) | Month 13 in respiratory |
| compared to the empty liposome control | symptom score |
| (ELC) + AZI + ETH on patient reported | |
| respiratory symptoms at Month 13 | |
| Secondary Objective: | Secondary Endpoint |
| To evaluate the efficacy of ALIS +AZI + ETH | |
| compared to ELC + AZI + ETH on the following: | |
| Durable culture | Proportion of subjects |
| conversion at Month 15 | achieving durable culture |
| | conversion at Month 15 |

The primary variable in the efficacy assessments of the study is the respiratory score of the QOL-B. The QOL-B questionnaire will be completed at study visits. Subjects will self-administer the questionnaire electronically. The computation of the respiratory score will be derived from selected items within the QOL-B instrument that will be determined based on results of qualitative work.

Microbiological assessment of sputum specimens will be used for secondary efficacy measurements.

During the study, pre-dose expectorated or induced sputum specimens (approximately 2 mL) are required at study visits. To improve the probability of obtaining a good sputum specimen, 2 sputum samples will be obtained from each subject at each assessment; subjects will provide 1 sputum sample on the day prior to the scheduled visit, and 1 sputum sample on the day of the visit prior to dosing with study drugs. At Screening, subjects will provide 1 sputum sample on the first day of the visit and 1 sputum sample during the week following the Screening visit. If a subject is unable to produce sputum spontaneously, sputum may be induced. If after induction, a subject is still unable to produce sputum despite reasonable efforts, this will be recorded as non-productive at that time point. At the scheduled visit, once sputum is collected (spontaneously or by induction), study drugs can be administered.

For microbiological assessment, sputum specimens will be cultured in broth (liquid) in addition to agar media (solid), and will be held for up to 6 weeks. A negative culture result will not be reported until after this time has transpired.

Growth of other bacteria due to co-infections (e.g., Pseudomonas aeruginosa, M. abscessus, other NTM) will be reported. Colony counts of MAC growth will be reported. Isolates of MAC will be identified using a commercial RNA probe (AccuProbe, Gen Probe, Inc., GenoType NTM-DR Ver 1.0, Hain Lifescience) and subsequently identified to species using molecular methodology. See Cousins et al. Multiplex PCR provides a low-cost alternative to DNA probe methods for rapid identification of Mycobacterium avium and Mycobacterium intracellulare. J Clin Microbiol. 1996; 34(9):2331-3, incorporated herein by reference in its entirety. Standard antibiotic sensitivity testing using MICs will be routinely performed on mycobacterial isolates. See Clinical and Laboratory Standards Institute. Laboratory detection and identification of mycobacteria; approved guideline. CLSI Document M48-A. Clinical and Laboratory Standards Institute, Wayne, PA: CLSI, 2008; Forbes et al. M48-A. Laboratory Detection and Identification of Mycobacteria; Approved Guideline. Clinical and Laboratory Standard Institute. 2008; 28(17), each of which is incorporated herein by reference in its entirety.

The MAC species and genotype at the Screening and/or Baseline sputum specimen will be identified by whole genome sequencing (Illumina Nextseq500™). In subjects who achieve sputum culture conversion and who subsequently have positive sputum culture(s) (broth or agar), typing by whole genome sequencing will also be conducted.

MAC culture assessment definitions of this study are listed in Table 10.

TABLE 10
MAC culture assessment definitions

| | |
|---|---|
| MAC culture | No MAC growth on agar media and broth media in all sputum cultures |
| negative | at a visit |
| Non- | Prior to culture conversion: visits where subjects were recorded as non- |
| productive | productive are considered MAC culture positive |
| | Post culture conversion: visits where subjects were recorded as non- |
| | productive are considered MAC culture negative |
| Culture | No MAC growth on agar media and broth media in all sputum cultures |
| conversion | at 2 consecutive visits. The date of conversion is defined by the date of |
| | the first of 2 consecutive negative cultures. |
| Durable | In subjects who achieve culture conversion at Month 12 (negative |
| culture | cultures for MAC at Month 11 and Month 12), and have negative |
| conversion | cultures at Month 13 and Month 15. |
| Recurrence: | Subsequent to culture conversion, a MAC positive culture that is the |
| Relapse | same species and genome as cultured at Screening/Baseline. The |
| | positive culture is defined as at least one positive culture in agar media |
| | or positive cultures in broth media in at least 2 consecutive visits. |
| Recurrence: | Subsequent to culture conversion, a MAC positive culture that is |
| New Infection | different than cultured at Screening/Baseline (different species, or same |
| | species but different genome). The positive culture is defined as at least |
| | one positive culture in agar media or positive cultures in broth media in |
| | at least 2 consecutive visits. |

Other outcome assessments included in the study are PROMIS Fatigue Short Form 7a (PROMIS F-SF 7a), PGIS Respiratory, and PGIS Fatigue, as described above, as well as actigraphy. Similar to that for the study in Example 1, the actigraphy of this study involves the use of the Philips ACTIWATCH SPECTRUM PRO actigraphy device worn by the subject. The device senses the activity of the wearer. The data collected will be downloaded at each in-clinic visit or during a home care visit.

### Statistical Analysis

The analyses of the primary and the key secondary endpoint will be performed on Intent-to-Treat (ITT-all randomized subjects) population. Superiority of ALIS treatment will be declared if significance at alpha of 0.05 (2-sided) is achieved. Only then will testing of the key secondary endpoint proceed (alpha of 0.05, 2-sided).

The analysis of the primary endpoint will assess differences in change from Baseline to Month 13 in PRO between the treatment groups. The testing and estimation of treatment effect together with corresponding 95% confidence intervals (CI) will utilize analysis of covariance (ANCOVA) with treatment group as factor, baseline score, and stratification variable (history of MAC lung infection, initial or subsequent) as factors.

The analysis of the key secondary endpoint of sputum conversion durability at Month 15 will utilize logistic regression model to estimate the odds ratio and corresponding 95% CI. Testing of difference in rates between treatment groups will be performed at alpha of 0.05 significance level, 2-sided, only if ALIS will be declared superior for the primary endpoint.

The analysis of safety data will be conducted on the safety analysis set (i.e., all randomly assigned subjects who receive any amount of study drug). Safety parameters presented for each treatment group will include the occurrence of AEs, the use of concomitant medications, and changes in clinical laboratory values (serum chemistry, hematology, and urinalysis), electrocardiograms (ECG), vital signs measurements, and physical examination findings (including body weight) between Baseline and EOT. Adverse events will be coded according to the latest version of the Medical Dictionary for Regulatory Activities (MedDRA) dictionary Version 23.0 or later. Summaries will be presented for all AEs, AEs determined by the Investigator to be treatment-related, serious adverse events (SAEs), and adverse events causing withdrawal from the study. Hematology, chemistry, and urinalysis values will be summarized by treatment group over time and by visit.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A method for treating a newly diagnosed and untreated Mycobacterium avium complex (MAC) lung infection in a patient in need of treatment, the method comprising:
(i) administering to the lungs of the patient for an administration period of at least about 6 months, a pharmaceutical composition comprising amikacin, or a pharmaceutically acceptable salt thereof, encapsulated in a plurality of liposomes, wherein the lipid component of the plurality of liposomes consists of dipalmitoylphosphatidylcholine (DPPC) and cholesterol, wherein administering to the lungs of the patient comprises aerosolizing the pharmaceutical composition via a nebulizer to provide an aerosolized pharmaceutical composition comprising a mixture of free amikacin, or a pharmaceutically acceptable salt thereof, and liposomal complexed amikacin, or a pharmaceutically acceptable salt thereof, and administering the aerosolized pharmaceutical composition via the nebulizer to the lungs of the patient once daily in a single dosing session during the administration period, and
(ii) administering to the patient during the administration period a macrolide antibiotic and ethambutol,
wherein the treating comprises improving a respiratory symptom score as measured by a patient reported outcome (PRO) during the administration period or subsequent to the administration period, compared to the respiratory symptom score of the patient prior to the administration period.

2. The method of claim 1, wherein the respiratory symptom score comprises the respiratory domain score of the Quality of Life Questionnaire-Bronchiectasis (QOL-B) or a modified version thereof.

3. The method of claim 2, wherein the respiratory symptom score comprises the respiratory domain score of a modified version of the Quality of Life Questionnaire-Bronchiectasis (QOL-B).

4. The method of claim 3, wherein the Quality of Life Questionnaire-Bronchiectasis (QOL-B) is modified to replace the term “bronchiectasis” with “lung condition”.

5. The method of any one of claims 1-4, wherein treating the respiratory symptom score further comprises the Patient Global Impression of Severity (PGIS) Respiratory score, and treating further comprises improving the PGIS Respiratory score during or subsequent to the administration period, as compared to the PGIS Respiratory score of the patient prior to the treatment.

6. The method of any one of claims 1-5, wherein the respiratory symptom score is improved during the administration period.

7. The method of any one of claims 1-6, wherein respiratory symptom score is improved at the end of the administration period.

8. The method of any one of claims 1-7, wherein the respiratory symptom score is improved about one month after the administration period.

9. The method of any one of claims 1-7, wherein the respiratory symptom score is improved at least about one month after the administration period.

10. The method of any one of claims 1-7, wherein the respiratory symptom score is improved from about one to about three months after the administration period.

11. The method of any one of claims 1-7, wherein the one or more respiratory symptoms for the patient are improved about three months after the administration period.

12. The method of any one of claims 1-7, wherein the respiratory symptom score is improved at least about three months after the administration period.

13. The method of any one of claims 1-7, wherein the respiratory symptom score is improved from about three to about twelve months after the administration period.

14. The method of any one of claims 1-13, wherein the treating further comprises improving one or more fatigue symptoms, as measured by a PROMIS Fatigue Short Form 7a score, for the patient during or subsequent to the administration period, as compared to the one or more fatigue symptoms of the patient prior to the administration period.

15. The method of any one of claims 1-14, wherein the treating further comprises improving one or more fatigue symptoms, as measured by a PGIS Fatigue score, for the patient during or subsequent to the administration period, as compared to the one or more fatigue symptoms of the patient prior to the administration period.

16. The method of claim 14 or 15, wherein the one or more fatigue symptoms for the patient are improved during the administration period.

17. The method of claim 14 or 15, wherein the one or more fatigue symptoms for the patient are improved at the end of the administration period.

18. The method of claim 14 or 15, wherein the one or more fatigue symptoms for the patient are improved at least about one month after the administration period.

19. The method of claim 14 or 15, wherein the one or more fatigue symptoms for the patient are improved from about one to about three months after the administration period.

20. The method of claim 14 or 15, wherein the one or more fatigue symptoms for the patient are improved at least about three months after the administration period.

21. The method of claim 14 or 15, wherein the one or more fatigue symptoms for the patient are improved from about three to about twelve months after the administration period.

22. The method of any one of claims 1-21, wherein the treating further comprises achieving a negative MAC sputum culture in the patient during or subsequent to the administration period.

23. The method of claim 22, wherein the treating comprises achieving a MAC sputum culture conversion in the patient during or subsequent to the administration period, wherein the MAC sputum culture conversion is defined as two consecutive negative MAC sputum cultures.

24. The method of claim 22, wherein the treating comprises achieving a MAC sputum culture conversion in the patient during or subsequent to the administration period, wherein the MAC sputum culture conversion is defined as three consecutive negative MAC sputum cultures.

25. The method of claim 23, wherein the two consecutive negative MAC sputum cultures are spaced about 30 days apart.

26. The method of claim 24, wherein the three consecutive negative MAC sputum cultures are spaced about 30 days apart from each other.

27. The method of any one of claims 23-26, wherein the patient achieves the MAC sputum culture conversion during the administration period.

28. The method of any one of claims 23-26, wherein the patient achieves the MAC sputum culture conversion at the end of the administration period.

29. The method of any one of claims 23-26, wherein the patient achieves the MAC sputum culture conversion at least about one month after the administration period.

30. The method of any one of claims 23-26, wherein the patient achieves the MAC sputum culture conversion from about one to about three months after the administration period.

31. The method of any one of claims 23-26, wherein the patient achieves the MAC sputum culture conversion about three months after the administration period.

32. The method of any one of claims 23-26, wherein the patient achieves the MAC sputum culture conversion at least about three months after the administration period.

33. The method of any one of claims 23-26, wherein the patient achieves the MAC sputum culture conversion from about three to about twelve months after the administration period.

34. The method of any one of claims 1-33, wherein the treating further comprises decreasing the length of time to achieve a first negative MAC sputum culture as compared to a patient with a newly diagnosed, untreated MAC lung infection administered the macrolide antibiotic and ethambutol, but not the pharmaceutical composition, for the same administration period.

35. The method of any one of claims 1-33, wherein the treating further comprises decreasing the length of time to achieve a MAC sputum culture conversion as compared to a patient with a newly diagnosed, untreated MAC lung infection administered the macrolide antibiotic and ethambutol, but not the pharmaceutical composition, for the same administration period, wherein the MAC sputum culture conversion is defined as two consecutive negative MAC sputum cultures.

36. The method of any one of claims 1-35, wherein the treating further comprises reducing the rate of recurrence of MAC for the patient, as compared to the rate of recurrence of MAC for a patient with a newly diagnosed, untreated MAC lung infection administered the macrolide antibiotic and ethambutol, but not the pharmaceutical composition, for the same administration period.

37. The method of claim 36, wherein the recurring MAC is of the same species and genome as the MAC prior to the treatment.

38. The method of claim 36, wherein the recurring MAC is of a different species, or of the same species but a different genome, as compared to the MAC prior to the treatment.

39. The method of any one of claims 1-38, wherein the treating further comprises improving the score of one or more of the QOL-B non-respiratory domains for the patient during or subsequent to the administration period, as compared to the score of the one or more of the QOL-B non-respiratory domains of the patient prior to the treatment.

40. The method of claim 39, wherein the score of the one or more of the QOL-B non-respiratory domains for the patient is improved during the administration period.

41. The method of claim 39, wherein the score of the one or more of the QOL-B non-respiratory domains for the patient is improved at the end of the administration period.

42. The method of claim 39, wherein the score of the one or more of the QOL-B non-respiratory domains for the patient is improved at least about one month after the administration period.

43. The method of claim 39, wherein the score of the one or more of the QOL-B non-respiratory domains for the patient is improved from about one to about three months after the administration period.

44. The method of claim 39, wherein the score of the one or more of the QOL-B non-respiratory domains for the patient is improved at least about three months after the administration period.

45. The method of claim 39, wherein the score of the one or more of the QOL-B non-respiratory domains for the patient is improved from about three to about twelve months after the administration period.

46. The method of any one of claims 1-45, wherein the treating further comprises improving the daily activity and sleep efficiency, as measured by a Philips ACTIWATCH SPECTRUM PRO actigraphy device, for the patient during or subsequent to the administration period, as compared to the daily activity and sleep efficiency experienced by the patient prior to the treatment.

47. The method of claim 46, wherein the daily activity and sleep efficiency for the patient is improved during the administration period.

48. The method of claim 46, wherein the daily activity and sleep efficiency for the patient is improved at least about one month after the administration period.

49. The method of claim 46, wherein the daily activity and sleep efficiency for the patient is improved from about one to about three months after the administration period.

50. The method of claim 46, wherein the daily activity and sleep efficiency for the patient is improved at least about three months after the administration period.

51. The method of claim 46, wherein the daily activity and sleep efficiency for the patient is improved from about three to about twelve months after the administration period.

52. The method of any one of claims 1-51, wherein the treating further comprises decreasing the rate of developing a MAC isolate with amikacin minimum inhibitory concentration (MIC)≥128 μg/mL for the patient, as compared to the rate of developing a MAC isolate with amikacin MIC≥128 μg/mL for a patient with a newly diagnosed, untreated MAC lung infection administered the macrolide antibiotic and ethambutol, but not the pharmaceutical composition, for the same administration period.

53. The method of any one of claims 1-52, wherein the administration period is from about six months to about 36 months.

54. The method of claim 53, wherein the administration period is from about six months to about 30 months.

55. The method of claim 53, wherein the administration period is from about six months to about 24 months.

56. The method of claim 53, wherein the administration period is from about six months to about 18 months.

57. The method of claim 53, wherein the administration period is from about six months to about 12 months.

58. The method of claim 53, wherein the administration period is from about 12 to about 36 months.

59. The method of claim 53, wherein the administration period is from about 12 to about 24 months.

60. The method of claim 53, wherein the administration period is about 12 months.

61. The method of claim 53, wherein the administration period is about 18 months.

62. The method of claim 53, wherein the administration period is about 24 months.

63. The method of any one of claims 1-52, wherein the administration period is at least about 12 months.

64. The method of any one of claims 1-63, wherein during the single dosing session, the aerosolized pharmaceutical composition is administered in less than about 15 minutes.

65. The method of any one of claims 1-63, wherein during the single dosing session, the aerosolized pharmaceutical composition is administered in about 10 minutes to about 14 minutes.

66. The method of any one of claims 1-65, wherein the pharmaceutical composition comprises from about 500 mg to about 650 mg amikacin, or pharmaceutically acceptable salt thereof.

67. The method of any one of claims 1-66, wherein the pharmaceutical composition comprises about 590 mg amikacin, or pharmaceutically acceptable salt thereof.

68. The method of any one of claims 1-67, wherein the amikacin or pharmaceutically acceptable salt thereof is amikacin sulfate.

69. The method of any one of claims 1-68, wherein the plurality of liposomes comprises unilamellar vesicles, multilamellar vesicles, or a mixture thereof.

70. The method of any one of claims 1-69, wherein the pharmaceutical composition comprises about 70 to about 75 mg/mL amikacin, or pharmaceutically acceptable salt thereof; about 32 to about 35 mg/mL DPPC; and about 16 to about 17 mg/mL cholesterol.

71. The method of any one of claims 1-69, wherein the pharmaceutical composition comprises about 70 mg/mL amikacin sulfate; about 30 to about 35 mg/mL DPPC; and about 15 to about 17 mg/mL cholesterol.

72. The method of claim 70 or 71, wherein the pharmaceutical composition further comprises about 1.5% (w/w) NaCl.

73. The method of any one of claims 1-72, wherein the pharmaceutical composition comprising amikacin or pharmaceutically acceptable salt thereof has a pH of about 6.5.

74. The method of any one of claims 1-73, wherein the macrolide antibiotic is azithromycin, clarithromycin, erythromycin, carbomycin A, josamycin, kitamycin, midecamycin, oleandomycin, solithromycin, spiramycin, troleandomycin, tylosin, roxithromycin, or a combination thereof.

75. The method of claim 74, wherein the macrolide antibiotic is azithromycin.

76. The method of claim 75, wherein azithromycin is administered orally once-daily.

77. The method of claim 76, wherein the azithromycin is in the form of a tablet.

78. The method of any one of claims 75-77, wherein the azithromycin is administered at a dosage of about 250 mg.

79. The method of claim 74, wherein the macrolide antibiotic is clarithromycin.

80. The method of claim 74, wherein the macrolide antibiotic is erythromycin.

81. The method of any one of claims 1-80, wherein ethambutol is administered orally once-daily.

82. The method of any one of claims 1-81, wherein the ethambutol is administered to the patient at a dosage of about 15 mg/kg.

83. The method of any one of claims 1-82, wherein the pharmaceutical composition comprising amikacin or pharmaceutically acceptable salt thereof is an aqueous dispersion.

84. The method of claim 83, wherein the volume of the pharmaceutical composition is from about 8 mL to about 10 mL.

85. The method of claim 83 or 84, wherein the volume of the pharmaceutical composition is about 8.4 mL.

86. The method of any one of claims 1-85, wherein the patient has a non-cavitary lung disease.

87. The method of any one of claims 1-86, wherein the patient has a non-cystic fibrosis lung disease.

88. The method of any one of claims 1-87, wherein the newly diagnosed and untreated MAC lung infection is defined as an untreated, initially diagnosed MAC lung infection based on a positive sputum culture for MAC.

89. The method of any one of claims 1-87, wherein the newly diagnosed and untreated MAC lung infection is defined as an untreated MAC lung infection diagnosed based on a newly positive sputum culture for MAC, subsequent to a previously diagnosed MAC lung infection based on a previous positive sputum culture for MAC, wherein the previously diagnosed MAC lung infection is treated, and the treatment of the previously diagnosed MAC lung infection was ceased when a negative sputum culture for MAC was achieved, and wherein the negative sputum culture for MAC returns to the newly positive sputum culture for MAC more than 6 months after the cessation of the treatment of the previously diagnosed MAC lung infection.

90. The method of claim 89, wherein the negative sputum culture for MAC returns to the newly positive sputum culture for MAC between about 6 months and about 12 months after the cessation of the treatment of the previously diagnosed MAC lung infection.

91. The method of claim 89 or 90, wherein the negative sputum culture for MAC returns to the newly positive sputum culture for MAC about 6 months to about 9 months after the cessation of the treatment of the previously diagnosed MAC lung infection.

92. The method of claim 89, wherein the treatment of the previously diagnosed MAC lung infection is ceased when a MAC sputum culture conversion in the patient is achieved, wherein the MAC sputum culture conversion is defined as two consecutive negative MAC sputum cultures, and the treatment of the previously diagnosed MAC lung infection is ceased when the second of the two consecutive negative MAC sputum cultures is achieved, and the second of the two consecutive negative MAC sputum cultures returns to the newly positive sputum culture for MAC at least 6 months after the cessation of the treatment of the previously diagnosed MAC lung infection.

93. The method of claim 92, wherein the second of the two consecutive negative MAC sputum cultures returns to the newly positive sputum culture for MAC about 6 months to about 12 months after the cessation of the treatment of the previously diagnosed MAC lung infection.

94. The method of claim 92 or 93, wherein the second of the two consecutive negative MAC sputum cultures returns to the newly positive sputum culture for MAC about 6 months to about 9 months after the cessation of the treatment of the previously diagnosed MAC lung infection.

95. The method of any one of claims 1-94, wherein about 96% to about 100% by weight of amikacin, or a pharmaceutically acceptable salt thereof, present in the composition is liposomal complexed prior to nebulization.

96. The method of any one of claims 1-94, wherein about 96% to about 99% by weight of amikacin, or a pharmaceutically acceptable salt thereof, present in the composition is liposomal complexed prior to nebulization.

97. The method of any one of claims 1-94, wherein about 96% to about 98% by weight of amikacin, or a pharmaceutically acceptable salt thereof, present in the composition is liposomal complexed prior to nebulization.

98. The method of any one of claims 1-97, wherein the weight ratio of the lipid component to amikacin, or a pharmaceutically acceptable salt thereof, is from about 0.6:1 (lipid:amikacin or a pharmaceutically acceptable salt thereof) to about 0.8:1 (lipid:amikacin or a pharmaceutically acceptable salt thereof).
